(19) United States
(12) Patent Application Publication
(10) Pub. No.: US 2004/0087559 A1
(43) Pub. Date: May 6, 2004

(54) Methods for prevention and treatment of cancer

(76) Inventors: Gary G. Schwartz, Winston-Salem, NC (US); Balakrishna L. Lokeshwar, Miami, FL (US); Tai C. Chen, Sudbury, MA (US); Lyman W. Whitlatch, Boston, MA (US); Michael F. Holick, Sudbury, MA (US)

Correspondence Address: Ted W. Whitlock, 5323 SW 38th Avenue, Ft. Lauderdale, FL, 33312, US

(21) Appl. No.: 10/695,509

(22) Filed: Oct. 28, 2003

Related U.S. Application Data
(63) Continuation of application No. 09/646,832, filed on Sep. 22, 2000, now abandoned.
Application No. 09/646,832 is a 371 of international application No. PCT/US99/06491, filed on Mar. 25, 1999.

(51) Int. Cl. A61K 31/59

(52) U.S. Cl. 514/167

(57) ABSTRACT

Methods for inhibiting, preventing, or treating cancer cell growth are disclosed. In one embodiment, the method comprises the step of administering to a patient an effective amount of a metabolic precursor of 1,25-dihydroxyvitamin D, or an analog or a derivative thereof, to increase levels of the metabolic precursor available to a target cell. In a preferred embodiment, the metabolic precursor is 25-hydroxyvitamin D.

### BACKGROUND OF THE INVENTION

1. Field of the Invention

The present invention relates to methods for the treatment of cancer in animals. In one embodiment, the method comprises the step of increasing the amount of a metabolic precursor of 1,25-dihydroxyvitamin D available to target cells or tissues in the animal. In another embodiment, the method comprises administering the gene expressing the enzyme 25-hydroxyvitamin D-1α-hydroxylase to a target cell to increase production of 1,25-dihydroxyvitamin D within the target cell.

The present invention also relates to a method for the treatment of hyperproliferative cell disorders and disorders of calcium and bone metabolism in animals, comprising administration of the gene expressing 25-hydroxyvitamin D-1α-hydroxylase to target cells or tissues of the animal.

2. Related Art

Definitions

The term “vitamin D” has been used in the literature to refer generally to a class of steroid hormones that possess anti-rachitic activity, including ergocalciferol (vitamin D₂), cholecalciferol (vitamin D₃), calcidiol (25-hydroxyvitamin D), calcitriol (1,25-dihydroxyvitamin D), and 20 or more other metabolites of vitamin D. However, the term “vitamin D” is used herein in its more restrictive sense to refer only to vitamin D₂ or vitamin D₃ in order to distinguish the useful metabolites which are part of this invention.

### 1,25-Dihydroxyvitamin D

The most biologically active vitamin D metabolite is 1,25-dihydroxyvitamin D (“1,25(OH)₂D”), also known as calcitriol. The synthesis of 1,25(OH)₂D in an animal, e.g., humans, begins with the cutaneous production of vitamin D after exposure to sunlight or ultraviolet (UV) radiation, or after the intestinal absorption of plant- and yeast-derived vitamin D₂ or animal-derived (typically from fish liver oil and fatty fish) vitamin D₃ obtained from the diet. To become biologically active, vitamin D must undergo two hydroxylation steps. See FIG. 1.

The first hydroxylation step occurs in the liver by hydroxylation at the 25th carbon position, forming 25-hydroxyvitamin D (“25(OH)D”), the major circulating metabolite of vitamin D. The second hydroxylation occurs in the kidney by hydroxylation at the 1α position, forming 1,25(OH)₂D, the hormonally active metabolite. Although the kidney is the major source of 1,25(OH)₂D, the enzyme 1α-hydroxylase (“1α-OHase”) that converts 25(OH)D to 1,25(OH)₂D is also present in several types of non-renal cells, e.g., activated macrophages and keratinocytes (Barbour, G. L., et al., N. Engl. J. Med. 305:440-443 (1981); and Bikle, D. D., et al., J. Clin. Invest. 78:557-566(1986)). However, 1α-OHase was not heretofore known to be present in prostatic cells.

The 1α-OHase enzyme has been cloned recently from mouse kidney, human kidney, and human keratinocytes (Takeyama, K., et al., Science 277:1827-1829 (1997); Fu, G. K., et al., Mol. Endo. 11:1961-1970 (1997); Monkawa, T., et al., Biochem. Biophys. Res. Comm. 239:527-533 (1997); Fu, G. K., et al., DNA and Cell Biology 16:1499-1507 (1997); Shinki, T., et al., Proc. Natl. Acad. Sci. USA 94:12920-12925 (1997); Brenza, H. L., et al., Proc. Natl. Acad Sci. USA 95:1387-1391 (1998); Murayama, A., et al., Biophys. Biochem. Res. Comm. 249:11-16 (1998); and Kong, X. F., et al., Bone 23 (5, Suppl):S186 (1998)). The cDNA was found to be identical for the 1α-OHase gene in the human kidney and keratinocytes (Kitanaka, S., et al., N. Engl. J. Med. 338:653-661 (1998); Fu, G. K., et al., Mol. Endo. 11:1961-1970 (1997); and Fu, G. K., et al., DNA and Cell Biology 16:1499-1507 (1997)). The 1α-OHase was also cloned from skin from patients with vitamin D dependent rickets type 1 (pseudovitamin D deficiency rickets). It was determined that several point mutations had occurred in the gene resulting in a defective 1α-OHase (Kitanaka, S., et al., N. Engl. J. Med. 338:653-661 (1998).

1,25(OH)₂D is able to carry out most of its biologic effects by interacting with a specific receptor known as the vitamin D receptor (VDR). VDR is present in tissues that are responsible for regulating calcium and bone metabolism including the small intestine, kidney and bone. In addition, a wide variety of tissues and cells not related to calcium and bone metabolism also possess VDR (Holick, M. F., in Primer on the Metabolic Bone Diseases and Disorders of Mineral Metabolism, M. J. Favus (ed.), 3rd Ed., Lippincott-Raven: Philadelphia (1996), pp. 74-81). These include, among others, brain, heart, gonads, breast, pancreas, skeletal muscle, smooth muscle, skin, monocytes, and activated T and B lymphocytes. In addition, several tumor cell lines, including promyelocytic leukemic cells, multiple myeloma cells, squamous cell carcinoma cells, prostate cancer cells, breast cancer cells, and basal cell carcinoma cells, possess VDR (Holick, M. F., Bone 17 (Suppl.) 107S-111S (1995)). Although the exact function of VDR in these tissues and cells is not well understood, it is known that some cells that possess VDR respond to 1,25(OH)₂D by decreasing their proliferative activity. In addition, some cells such as keratinocytes are also induced to terminally differentiate. This has given rise to the use of 1,25(OH)₂D and its analogs for treating hyperproliferative skin diseases such as psoriasis, and has suggested the possibility for treating some cancers such as breast, colon, and prostate (Holick, M. F., Bone 17 (Suppl.):107S-111S (1995); Gross, C, et al., in Vitamin D, D. Feldman et al. (eds.), Academic Press: New York (1997), pp. 1125-1139).

### Treatment of Cancer

Cancer is a group of diseases characterized by the uncontrolled growth and spread of abnormal cells. One frequently-occurring cancer is adenocarcinoma of the prostate gland, which is the most commonly diagnosed malignancy in American males, excluding skin cancer. Approximately 317,000 new cases of prostate cancer were diagnosed in the United States in 1996 (Parker, S. L., et al., Cancer J. Clin. 46:5-27 (1996)), accounting for 41% of newly diagnosed malignancies, excluding basal cell and squamous cell carcinomas of the skin. This may actually underestimate the problem because clinically silent prostate cancer is very common.

A unique feature of prostate cancer is the high prevalence of “incidental” or “subclinical” cases identified at autopsy. Autopsy data indicate that about 30% of men over the age of 50 have histological prostate cancer, and the prevalence of these incidental cancers reaches 60% in men over the age of 80. Histologically, these incidental cancers are indistinguishable from prostate cancers that are potentially life-threatening and are considered to be an earlier stage in their natural history. Unlike prostate cancer mortality rates, incidental prostate cancer is ubiquitous among elderly men regardless of ethnicity or geographic location.

Although prostate carcinoma is generally a slow growing malignancy, mortality from the disease is nonetheless considerable, representing about 15% of all cancer deaths in 1996, which makes prostate cancer the second leading cause of cancer death among U.S. males. (Parker, S. L., et al., Cancer J. Clin. 46:5-27 (1996)). As a result, prostate cancer has rapidly become a major public health concern not only in the United States, but worldwide. See Gross, C., et al., in Vitamin D, D. Feldman et al. (eds.), Academic Press: San Diego, Calif. (1997), pp. 1125-1139. Interestingly, Prostate cancer mortality rates in the contiguous United States are inversely correlated with levels of ultraviolet (UV) radiation, which produces vitamin D in the skin.

As suggested above, calcitriol is now known to play an important role in the regulation of cell growth and differentiation (Walter, M. R., Endocr. Rev. 13:719-764 (1992)). For example, it has been shown that 1,25(OH)₂D can modulate cell proliferation and differentiation in prostate cells. Supporting evidence includes the ubiquitous presence of receptors for 1,25(OH)₂D (vitamin D receptors, or VDRs) in human prostatic cells and the anti-proliferative and pro-differentiating effects of 1,25(OH)₂D on these cells in vitro and in vivo (Schwartz, G. G., Anticancer Res. 14:1077-1082 (1994)). Apparently, 1,25(OH)₂D maintains the differentiated phenotype of prostatic cells. Low levels of 1,25(OH)₂D can therefore increase the risk for life-threatening prostate cancer.

It would appear that making 1,25(OH)₂D available to target cells or organs would reduce the risk of cancer. However, increasing 1,25(OH)₂D levels is subject to a plurality of obstacles. First, systemic levels of 1,25(OH)₂D in normal individuals are very tightly regulated metabolically. An increase in systemic 1,25(OH)₂D levels is generally not correlated with an increase in systemic levels of vitamin D or certain of its metabolites, including 25(OH)D, the immediate metabolic precursor of 1,25(OH)₂D. Thus, a mechanism by which UV radiation or vitamin D could result in increased exposure to 1,25(OH)₂D by prostatic cells was previously unclear. Prostatic cells, even though known to have 1,25(OH)₂D receptors, were previously thought to be modulated only by systemic 1,25(OH)₂D which was produced in and highly regulated by kidney 1α-OHase using 25(OH)D as a substrate. Because of this highly regulated production of 1,25(OH)₂D by the kidneys, it was well accepted that increasing systemic 25(OH)D would not produce proportionately higher systemic levels of 1,25(OH)₂D.

It was also previously unknown how to provide effective amounts of 1,25(OH)₂D at the cellular level without risking undesired, and potentially unsafe, side effects to the patient. For example, systemic 25(OH)D levels can be increased by in situ production of vitamin D through sun or UV exposure, due to the regulation of 1,25(OH)₂D produced from 25(OH)D. Thus, although extensive exposure to sunlight or UV radiation will elevate serum levels of 25(OH)D, serum levels of 1,25(OH)₂D will generally not be elevated to a desired antiproliferative level. Thus, reaching a beneficial level of 1,25(OH)₂D was not known to be possible from exposure to the sun or UV radiation, even in amounts that are considered by those in the art to be unsafe or excessive. Certain risks, e.g., skin cancer, inter alia, are associated with such excessive sun or UV exposure.

It has recently been suggested that vitamin D dietary supplementation may lower the risk of prostate cancer (Naitoh, J., et al., Prostate Cancer and Prostatic Diseases 1:48-53 (1997)). However, administering vitamin D as a dietary supplement is recommended to be performed under the supervision of a physician due to the risk of producing vitamin D toxicity, of which the negative side effects can include kidney failure, hypercalcemia, coma, and death.

Vitamin D supplementation therapy is also disadvantageous in view of its slow metabolic conversion to 25(OH)D. Therefore, reaching effective levels of 25(OH)D by administering supplemental vitamin D can take weeks or months using a conservative dosing regimen. Conversely, if those vitamin D levels are too high, it can take months to return to normal levels of 25(OH)D after ceasing the administration or reducing the dosage of vitamin D due to the natural storage of vitamin D in body fat. Moreover, administration of 1,25(OH)₂D itself, carries its own risks of producing hypercalcemia, which has been well-documented.

Multiple studies that have shown that several lines of cancer cells including colon, melanoma, prostate and breast that have a VDR, when incubated with 1,25(OH)₂D, decreased their proliferation (Holick, M. F., Bone 17 (2 Suppl.): 107S-111S (1995); Halline, A. G., et al., Endocrinology 134:1710-1717 (1994); Konety, B. R., et al., Cell Growth & Differentiation 7:1563-1570 (1996); and Kivineva, M., et al., J. Steroid Biochem. Mol. Biol. 66:121-127 91998)). Furthermore, human skin cells (Bikle, D. D., et al., Biochemistry 25:1545-1548 (1986)) and normal prostate cells and prostate cancer cells have been shown to have the capacity to convert 25(OH)D to 1,25(OH)₂D (Schwartz, G. G., et al., Cancer Epidemiol. Biomark & Prev. 7:391-395 (1998)). Thus, it is believed that when such cells are exposed to 25(OH)D, that the 25(OH)D is converted by the cells' 1α-OHase to 1,25(OH)₂D. The internally produced 1,25(OH)₂D then will interact with the cell's VDR to regulate the cell's proliferative activity.

As described above, 1,25(OH)₂D is considered to the biologically active form of vitamin D. Its principal physiologic function is on calcium and bone metabolism. It stimulates intestinal calcium transport and initiates mobilization of calcium stores from bone. Patients with kidney failure who are unable to make 1,25(OH)₂D develop hypocalcemia, secondary hyperparathyroidism, and the bone disease, renal osteodystrophy. Two rare genetic disorders also are associated with an inherited defect in the renal 1α-OHase. Vitamin D dependent rickets type I is an inborn error where there is a defect in the 1α-OHase enzyme. In X-linked hypophosphatemic rickets, there is a defect in the regulation of the 1α-OHase. The elderly, with osteoporosis, have a defect in their ability to upregulate the 1α-OHase (Holick, M. F., in Primer on the Metabolic Bone Diseases and Disorders of Mineral Metabolism, M. J. Favus (ed.), 3rd Ed., Lippincott-Raven: Philadelphia (1996). pp. 74-81).

### SUMMARY OF THE INVENTION

It has not been heretofore described that administering a vitamin D metabolite and 1,25(OH)₂D precursor to a patient can prevent cell proliferation, invasive cancer, or metastasis of cancer or tumor cells of an organ, e.g., the prostate, colon, or breast, while reducing the risk of vitamin D toxicity and hypercalcemia.

It has also not been heretofore described that administration of the 1α-OHase gene using gene therapy techniques could be used to treat or prevent cancer or any disease in which 1,25(OH)₂D₃ can influence cell growth and maturation, including hyperproliferative skin disorders such as psoriasis or actinic keratoses, or that it could also be used to treat disorders in calcium and bone metabolism, as a safer alternative to exogenous administration of 1,25(OH)₂D₃.

In addition, there was previously no known method for determining the risk for development of invasive cancer nor to predict the success rate by a particular treatment by measuring in an organ levels of cellular components, e.g., vitamin D, vitamin D metabolites, the enzymes producing those metabolites, or the activity of those enzymes.

Such methods are advantageously provided by the subject invention as described hereinbelow.

The subject invention concerns novel methods for prevention, treatment or testing for certain types of cancer, in particular prostate cancer, employing the advantages of a metabolite of vitamin D, analogs or derivatives of the vitamin D metabolite, or enzymes involved in the metabolism of vitamin D.

The invention provides a method for locally increasing concentrations of 1,25-dihydroxyvitamin D (1,25(OH)₂D) in a cell of a target organ so that the anti-proliferative, anti-invasive, antimetastatic and pro-differentiating action of 1,25(OH)₂D can be provided to those cells. Specifically, one embodiment of the subject invention concerns a method for increasing 1,25(OH)₂D concentrations available to the target organ by administering to a patient a metabolic precursor of 1,25(OH)₂D wherein the precursor does not have the side effects or risks of skin cancer associated with sun or ultraviolet (UV) radiation exposure, or the side effects or risks of vitamin D toxicity associated with administering high doses of vitamin D or 1,25(OH)₂D.

In a preferred embodiment, the invention comprises administering an effective amount of a vitamin D metabolite to a patient in need of therapeutic or prophylactic treatment of a tumor or cancer, wherein the administration of the metabolite provides to a patient a substantially lower risk of skin cancer, vitamin D toxicity, or hypercalcemia than alternative methods, e.g, exposure to UV radiation, or administration of excessively high doses of vitamin D or 1,25(OH)₂D which can cause such undesired side effects.

Advantageously, the subject method can be used to prophylactically or therapeutically treat a patient at risk of or having certain subclinical or clinical cancer. The method includes preventing proliferation, invasion, or metastasis of the tumor or cancer cells by administering vitamin D or a metabolite of vitamin D, or an analog, derivative salt, or functional equivalent of the metabolite which can be metabolically converted to 1,25(OH)₂D or a functional equivalent thereof. The subject treatment preferably comprises administering to a patient having subclinical, clinical, or minimal residual cancer which is responsive to 1,25(OH)₂D, an effective amount of 25(OH)D. Minimal residual cancer refers to detectable tumor cells in post-treatment patients, e.g., post-prostatectomy or post-radiation therapy, wherein the primary tumor has been eliminated or the organ has been removed from the body, but tumor or cancer cells, likely mobilized to a different organ or present in the bloodstream, are still detectable.

In a further preferred embodiment, the subject method for prophylactic or therapeutic treatment of cancer comprises administering an effective amount of 25(OH)D, or an analog, derivative, salt, or functional equivalent thereof, to a patient having subclinical, clinical, or minimal residual cancer of an organ, i.e., the “target organ,” wherein the target organ or cells derived from that target organ can convert 25(OH)D or its analog, derivative, salt, precursor, or functional equivalent, to 1,25(OH)₂D, or its functional equivalent. Organ cells having the enzyme 1α-OHase can locally convert 25(OH)D or an analog thereof to 1,25(OH)₂D or its corresponding analog. For example, certain prostatic cells have been shown to have 1α-OHase and the capability to convert 25(OH)D to 1,25(OH)₂D. Accordingly, the subject method can be applied to prostate cancer patients having subclinical or clinical disease; patients at risk of developing prostate tumors (e.g., persons with no clinical evidence of prostate cancer), or patients having metastatic disease, e.g., minimal residual cancer, resulting from mobilization of cancerous prostatic cells to other organs or tissues.

Alternatively, the subject method can be used for treating a tumor in any organ wherein the cells of that organ can convert a metabolic precursor of 25(OH)D to 1,25(OH)₂D, or its functional equivalent, and be responsive to the antiproliferative, anti-invasive, antimetastatic, or pro-differentiating effects of 1,25(OH)₂D, or its functional equivalent. Thus, a cell having a vitamin D receptor (VDR) and capable of producing the enzyme, 1α-OHase, can respond to such treatment. Further, a cell normally deficient in VDR or 1α-OHase can be genetically engineered, e.g., transgenically altered with an appropriate gene or promotor to induce transcription of the gene and production of the resultant protein(s), such that the cell is capable of responding to treatment according to the subject invention.

The subject invention thus offers a solution to the problem of reducing the risk of invasive (i.e., life-threatening) cancer, including invasive prostate cancer. Therapy with 25(OH)D or an analog, derivative, salt, or functional equivalent thereof can be useful to many men at various stages of the natural history of prostate cancer. These include the millions of men who presently have subclinical prostate cancer: men on “watchful waiting,” and men who have been treated for prostate cancer by therapy with curative intent, but who may still have prostatic cancer cells remaining in their bodies. It also may be useful to men without prostate cancer, either subclinical or clinical, to reduce the risk of developing cancer. One form of subclinical prostate cancer is also known as “prostatic intraepithelial neoplasia” or PIN.

It should also be possible to inhibit cell proliferation in cancer cells which contain the VDR and which respond to 1,25(OH)₂D by decreasing their proliferation, by introducing into the cancerous cells the gene coding for the enzyme 1α-OHase and allowing the cells to produce 1,25(OH)₂D internally. This gene therapy method could therefore be used to treat these cancers. Further, if the gene encoding the VDR were co-administered with the 1α-OHase gene to cells lacking the VDR, this method might impart to such cells a response to 1,25(OH)₂D₃, making this method useful in the treatment of cancers normally unresponsive to 1,25(OH)₂D₃.

There would be several advantages to this method. For example, exogenous administration of 1,25(OH)₂D₃ and its analogs has the potential for causing hypercalcemia (Holick, M. F., in Primer on the Metabolic Bone Diseases and Disorders of Mineral Metabolism, M. J. Favus (ed.), 3rd Ed., Lippincott-Raven: Philadelphia (1996), pp. 74-81; Holick, M. F., Bone 17 (2 Suppl.): 107S-111S (1995); and Gross, C., et al., in Vitamin D, D. Feldman et al. (eds.), Academic Press: San Diego, Calif. (1997), pp. 1125-1139), which would be mitigated or eliminated by incorporating the 1α-OHase gene into the cancer cell and allowing it to make 1,25(OH)₂D internally. The co-administration of the VDR gene will either impart or enhance the cell response to the antiproliferative activity of the endogenously produced 1,25(OH)₂D. Once made, 1,25(OH)₂D is likely to be degraded in the cell after its use thereby eliminating any potential toxicity.

In a preferred embodiment, the invention provides a method of inhibiting proliferation of tumor cells and/or inducing differentiation of tumor cells in an animal, comprising introducing into the cells in need thereof a polynucleotide construct comprising a gene expressing 1α-OHase, whereby 1,25(OH)₂D or an analog thereof is produced and the proliferation of the cell is inhibited. In a preferred embodiment, the invention further comprises introducing into the tumor cells a polynucleotide construct comprising a gene expressing the vitamin D receptor. In this case, the gene expressing 1α-OHase and the gene expressing the vitamin D receptor can be on either the same DNA construct or on separate DNA constructs.

In a particularly preferred embodiment, this aspect of the invention provides a method for treating or preventing prostate cancer, breast cancer, skin cancer, colon cancer, lung cancer, leukemia, lymphoma, or other cancers that can respond to the antiproliferative and prodifferentiating actions of endogenously produced 1,25(OH)₂D or an analog of 1,25(OH)₂D that is provided by the cell undergoing gene therapy with DNA constructs containing the 1α-OHase gene, with or without the VDR gene.

In another embodiment, an animal being treated by gene therapy as described herein may also be treated substantially simultaneously with 25(OH)D or an analog, derivative, salt or functional equivalent thereof to augment the response to the gene therapy.

In addition, the invention provides a method of testing for cancer risk of a patient. By measuring levels of certain cellular components in a particular organ wherein the measured component is involved in vitamin D metabolism, vitamin D, a metabolite thereof (e.g., 25(OH)D, or its analogs), an enzyme capable of converting a vitamin D metabolite to 1,25(OH)₂D or the activity of that enzyme, the relative risk of developing life-threatening cancer in that organ can be determined. For example, the 1α-OHase enzyme is capable of converting 25(OH)D to 1,25(OH)₂D. Therefore, measuring 1α-OHase levels or the activity of the 1α-OHase enzyme in a cellular sample from a target organ, e.g., prostate, can be used to determine the risk of developing cancer in that organ or an organ comprising a cancerous cell from that organ.

Standard molecular biology techniques which are well-known in the art can also be used for measuring genetic polymorphisms in the genes encoding the 1α-OHase enzyme or VDRs and correlating allelic differences with risk of tumor development in the individual tested.

The invention also provides a method of predicting the rate of successful treatment of a particular cancer in a patient by determining the local levels of certain cellular components in the vitamin D metabolic pathway, such as vitamin D or its metabolites, in an organ, e.g., the prostate, or by determining levels of an enzyme, e.g., 1α-OHase, capable of converting a metabolic precursor into 1,25(OH)₂D. Measuring activity of the enzyme can also be useful for indirectly measuring enzyme levels and thus be predictive of treatment success.

The invention also provides a method of treating a hyperproliferative skin disorder in an animal, comprising introducing into skin cells of the animal in need thereof a polynucleotide construct containing a gene expressing 1α-OHase, whereby 1,25(OH)₂D or an analog thereof is produced and the proliferation of the cell is inhibited. In another embodiment, the invention further comprises introducing into said cells a polynucleotide construct containing a gene expressing the vitamin D receptor. In this embodiment of the invention, the gene expressing 1α-OHase and the gene expressing the vitamin D receptor can be on the same DNA molecule of the construct or on separate DNA molecules of said constructs.

More specifically, the invention can be used to treat actinic keratoses, a pre-skin cancer, or ichthyosis. Introduction of the gene coding for 1α-OHase effects an increase of the amount of 1,25(OH)₂D₃ within the cell, thereby decreasing proliferative activity and inducing the cells to differentiate into normal morphology.

The invention is also directed to a method to treat hyperproliferative disorders of the skin such as psoriasis. Patients with psoriasis respond to topical and oral 1,25(OH)₂D. However, there is concern about the potential toxicity (Holick, M. F., in Primer on the Metabolic Bone Diseases and Disorders of Mineral Metabolism, M. J. Favus (ed.), 3rd Ed., Lippincott-Raven: Philadelphia (1996), pp. 74-81; and Holick, M. F., Bone 17 (2 Suppl.):107S-111S (1995)). Thus, the invention is a reasonable alternative treatment for psoriasis, whereby the cells are induced to make or boost their production of 1,25(OH)₂D₃ which, in turn, interact with the VDR and decrease the proliferative activity and enhance differentiation, making the skin cells normal. This treatment can also be applied to other hyperproliferative skin disorders such as ichthyosis.

The invention also provides a method of treating a disorder in calcium and bone metabolism in an animal, comprising introducing into cells of the animal in need thereof a polynucleotide construct containing a gene expressing 1α-OHase, whereby 1,25(OH)₂D or an analog thereof is produced and the disorder is treated. In one embodiment, the invention further comprises introducing into said cells a polynucleotide construct containing a gene expressing the vitamin D receptor. In this embodiment, the gene expressing 1α-OHase and the gene expressing the vitamin D receptor can be on the same DNA construct or on separate DNA constructs.

This embodiment of the invention provides a method for treating disorders in calcium and bone metabolism such as renal osteodystrophy or a metabolic bone disease. The invention also provides a method of treating vitamin D-dependent rickets type I, X-linked hypophosphatemic rickets, vitamin D-dependent rickets type II, and osteoporosis, as well as such disorders in calcium and bone metabolism as low blood serum calcium due to hypoparathyroidism.

The present invention also provides a method of treating or preventing hair loss in an animal, comprising introducing into hair cells in need thereof a polynucleotide construct containing a gene expressing 1α-OHase, whereby 1,25(OH)₂D or an analog thereof is produced and the hair loss is treated. In one embodiment, the invention further comprises introducing into said cells a polynucleotide construct containing a gene expressing the vitamin D receptor. In this embodiment, the gene expressing 1α-OHase and the gene expressing the vitamin D receptor can be on the same DNA construct or are on separate DNA constructs.

The invention can therefore be used to treat or prevent hair loss resulting from conditions such as alopecia androgenetica, female pattern baldness, and chemotherapy-induced alopecia.

The invention also provides a method of enhancing wound healing in all animal, comprising introducing into cells of the wound in need thereof a polynucleotide construct containing a gene expressing 1α-OHase, whereby 1,25(OH)₂D or an analog thereof is produced and the healing of the wound is enhanced. In one embodiment, the invention further comprises introducing into said cells a polynucleotide construct containing a gene expressing the vitamin D receptor. In this embodiment, the gene expressing 1α-OHase and the gene expressing the vitamin D receptor can be on the same DNA construct or are on separate DNA constructs.

The invention also provides a method of treating benign prostatic hyperplasia in an animal, comprising introducing into cells exhibiting benign prostatic hyperplasia in need thereof a polynucleotide construct containing a gene expressing 1α-OHase, whereby 1,25(OH)₂D or an analog thereof is produced and the benign prostatic hyperplasia is treated. In this embodiment, the invention may further comprise introducing into said cells a polynucleotide construct containing a gene expressing the vitamin D receptor. In this embodiment of the invention, the gene expressing 1α-OHase and the gene expressing the vitamin D receptor can be on the same DNA construct or are on separate DNA constructs.

The invention also provides a method of treating an autoimmune disease in an animal, comprising introducing into cells of the animal in need thereof a polynucleotide construct comprising a gene expressing 25-hydroxyvitamin D-1α-hydroxylase, whereby 1,25-dihydroxyvitamin D or an analog thereof is produced and the autoimmune disease is treated. In one embodiment, the invention may further comprise introducing into said cells a polynucleotide construct containing a gene expressing the vitamin D receptor. In this embodiment, the gene expressing 1α-OHase and the gene expressing the vitamin D receptor can be on the same DNA construct or are on separate DNA constructs.

The invention can be used to treat autoimmune diseases such as diabetes mellitus type 1, multiple sclerosis, rheumatoid arthritis, and psoriatic arthritis.

In addition, the invention provides for an isolated polynucleotide molecule comprising a 1α-OHase regulatory sequence or a complement thereof. In one embodiment, the 1α-OHase regulatory sequence or a complement thereof comprises a polynucleotide selected from the group consisting of: (a) a polynucleotide comprising a nucleotide sequence at least 90% identical to the nucleotide sequence depicted in SEQ ID NO. 3; and (b) a polynucleotide that hybridizes under stringent conditions to the polynucleotide of (a) or the complement thereof. In other embodiments, the 1α-OHase regulatory sequence or complement thereof comprises a nucleotide sequence that is 95%, 97%, 98%, or 99% identical to the sequence depicted in SEQ ID NO. 3, or comprises the nucleotide sequence depicted in SEQ ID NO. 3. The invention also provides for an isolated polynucleotide molecule further comprising a nucleotide sequence coding for 1α-OHase that is 95%, 97%, 98%, or 99% identical to the sequence depicted in SEQ ID NO. 1, or one that comprises the nucleotide sequence depicted in SEQ ID NO. 1.

### BRIEF DESCRIPTION OF THE FIGURES

FIG. 1 shows a diagrammatic representation of vitamin D metabolism in the body. Well-known metabolism steps are shown by solid arrows; synthesis of 1,25(OH)₂D from 25(OH)D by the prostate is shown by open arrows.

FIGS. 2-5 show a high performance liquid chromatography elution profile of tritium activity of lipid extracts from cultured normal human prostate primary cultures or BPH cultures incubated with [³H]-25(OH)D in the absence or presence of the P450 inhibitor clotrimazole.

FIG. 2 shows second passage of normal human prostate cells incubated with nonradioactive 25(OH)D₃ (50 nM), [³H]-25(OH)D₃ (0.91 μCi/nmol) and 1,2-dianilinoethane (DPPD) (10 μM) at 37° C. for two hours in the absence of clotrimazole.

FIG. 3 shows second passage of normal human prostate cells incubated with nonradioactive 25(OH)D₃ (50 nM), [³H]-25(OH)D₃ (0.91 μCi/nmol) and DPPD (10 μM) at 37° C. for two hours in the presence of the P450 enzyme inhibitor, clotrimazole (20 μM).

FIG. 4 shows second passage of prostate cells derived from BPH cultures incubated with nonradioactive 25(OH)D₃ (50 nM), [³H]-25(OH)D₃ (0.91 μCi/nmol) and DPPD (10 μM) at 37° C. for two hours in the absence of clotrimazole.

FIG. 5 shows second passage of prostate cells derived from BPH cultures incubated with nonradioactive 25(OH)D₃ (50 nM), [³ H]-25(OH)D₃ (0.91 μCi/nmol) and DPPD (10 μM) at 37° C. for two hours in the absence of clotrimazole.

FIG. 6 depicts a graph showing the effect of 25(OH)D₃ concentration on the proliferative activity of normal human keratinocytes transfected with 1α-OHase cDNA. Cultured human keratinocytes transfected with a vector containing the 1α-OHase cDNA as described were incubated with 25(OH)D₃ at different concentrations (1 nM, 10 nM, 100 nM) for 18 hours. [³H]thymidine incorporation was measured as described. ●-: [³H]thymidine incorporation of cells not transfected (“Control”); ∘-∘: [³H]thymidine incorporation of cells transfected with vector containing 1α-OHase cDNA (“1α-OHase transfection”).

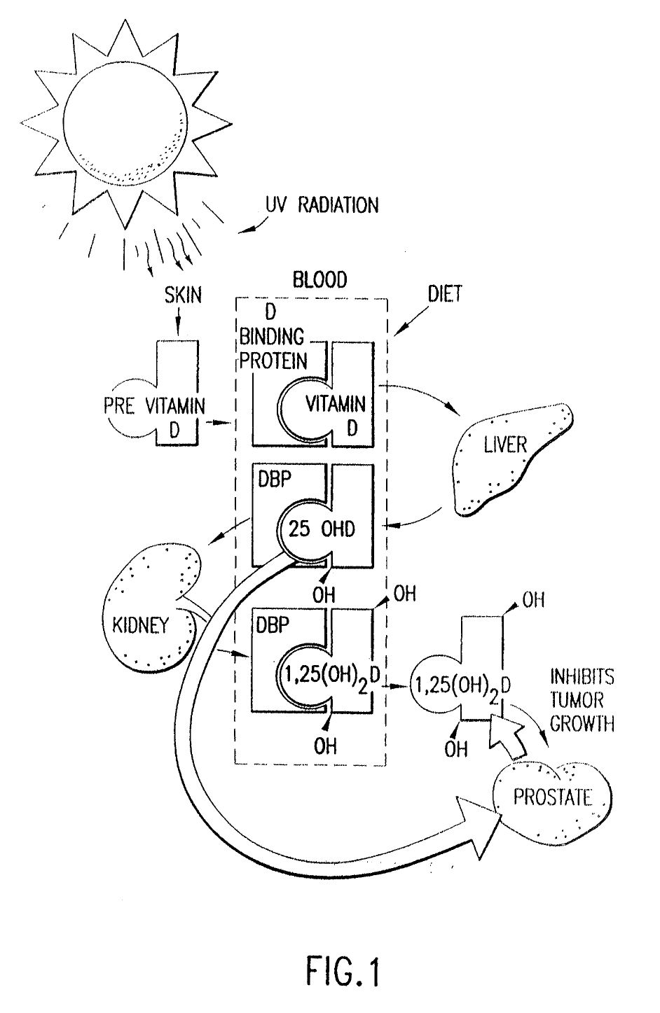

### DETAILED DESCRIPTION OF THE PREFERRED EMBODIMENTS

The subject invention concerns novel methods for preventing or treating cell proliferation, invasiveness, or metastatic potential, or for promoting cellular differentiation. The subject invention further concerns a method for testing for cancer, as well as a method for predicting the success of treatment for cell proliferation or cancer.

### Administration of Vitamin D Metabolite Compounds

One aspect of the subject invention comprises increasing local cellular levels of 1,25(OH)₂D. Specifically, one embodiment of the subject invention concerns preventing or treating cell proliferation, invasiveness, or metastasis, or promoting cellular differentiation by administering an effective amount of a vitamin D metabolite which can be metabolically converted by the target cells to 1,25(OH)₂D.

Preferably, the vitamin D metabolite used in accordance with the subject invention does not cause an increased risk of skin cancer (as compared to sun or ultraviolet (UV) ray exposure), vitamin D toxicity (as compared to supplemental excessive vitamin D administration), and does not significantly contribute to hypercalcemia (as compared to administering 1,25(OH)₂D). More preferably, the subject invention comprises administering an effective amount of 25(OH)D, or an analog, derivative, salt, or functional equivalent thereof to prevent the proliferation, invasion, or metastasis of tumor or cancer cells in a particular organ, or to promote differentiation of cells in, or derived from, that organ. The compound 25(OH)D is commercially available.

The normally observed concentration of 25(OH)D in serum is about 20-150 nmol/L (8-60 ng/ml). However, circulating concentrations of up to 250 nmol/L (100 ng/ml) 25(OH)D have commonly been observed in lifeguards after a full summer of exposure to sunlight and is considered to be normal (Holick, M. F., J. Nutrition, Suppl. 120:1464-1469(1990)). Concentrations of about 350 nmol/L or more are considered to be dangerous to the health of the individual. Hypovitaminosis D, i.e., a deficiency in serum 25(OH)D, is a condition defined when serum levels fall below about 25 nmol/L (10 ng/ml). Thus, an effective amount of 25(OH)D administered into the target organ would be any amount which, when administered, increases local cellular levels of 25(OH)D, but maintains serum levels of 25(OH)D within this “normal” range. Preferably, 25(OH)D levels are increased in the target organ substantially above 25 nmol/L but less than 250 nmol/L. More preferably, 25(OH)D levels are increased to between about 50 and 150 nmol/L.

Normal serum levels of 1,25(OH)₂D range between about 38-144 pmol/L (16-60 pg/ml). Thus, an alternative determination of an effective amount of 25(OH)D administered in accordance with the method of the subject invention is to administer an amount which raises the level of 25(OH)D toward the high end of its normal range in the target organ, but which does not raise systemic 1,25(OH)₂D above the high end of its normal range. For example, 25(OH)D levels preferably can be raised to increase intra-organ levels to between about 25 and 150 nmol/L, but where systemic 1,25(OH)₂D levels remain less than 145 pmol/L. More preferably, 1,25(OH)₂D levels remain below 125 nmol/L when 25(OH)D is administered to a target cell, organ, or tissue.

Useful analogs, derivatives, or salts of 25(OH)D include alkylated, glycosylated, arylated, halogenated, or hydroxylated 25(OH)D, orthoesters of 25(OH)D, or pharmaceutical salts of 25(OH)D. These analogs, derivatives, or salts can be synthesized or otherwise manufactured by chemical procedures which are well-known and readily available to those of ordinary skill in the art. The vitamin D analogs can be obtained according to the methods disclosed in U.S. Pat. Nos. 5,508,392, 5,457,217, 5,414,098, 5,384,313, 5,373,004, 5,371,249, 5,430,196, 5,260,290, 5,393,749, 5,395,830, 5,250,523, 5,247,104, 5,397,775, 5,194,431, 5,281,731, 5,254,538, 5,232,836, 5,185,150, 5,321,018, 5,086,191, 5,036,061, 5,030,772, 5,246,925, 4,973,584, 5,354,744, 4,927,815, 4,857,518, 4,851,401, 4,851,400, 4,847,012, 4,755,329, 4,940,700, 4,619,920, 4,594,192, 4,588,716, 4,564,474, 4,552,698, 4,588,528, 4,719,204, 4,719,205, 4,689,180, 4,505,906, 4,769,181, 4,502,991, 4,481,198, 4,448,726, 4,448,721, 4,428,946, 4,411,833, 4,410,515, 4,367,177, 4,336,193, 4,360,472, 4,360,471, 4,307,231, 4,307,025, 4,358,406, 4,305,880, 4,279,826, and 4,248,791.

The term “functional equivalent” is used to refer to any compound which can be used as a substrate for 1α-OHase or otherwise can be converted to 1,25(OH)₂D or converted to a compound which can bind to or activate the 1,25(OH)₂D receptor (VDR) such that any one of the beneficial properties of 1,25(OH)₂D, namely inhibition of invasiveness, proliferation, metastasis, or promotion of cell differentiation, are effected. See, for example, U.S. Pat. Nos. 5,167,953; 5,422,099; 5,794,248; and 5,395,829.

It would also be understood that the subject invention can be carried out by use of pharmaceutical compositions which comprise a 1,25(OH)₂D metabolic precursor or analog or derivative thereof and, e.g., 25(OH)D, a pharmaceutically acceptable carrier.

The subject method of administering a metabolic precursor of 1,25(OH)₂D to a patient has been shown to be effective in producing 1,25(OH)₂D by prostatic cancer cells. In particular, the subject method has been shown to be effective in two well-characterized human prostate cancer cell lines, DU 145 and PC-3, and two primary cultures of cells, NP96-5 and BPH96-11, derived from noncancerous human prostates. The NP96-5 culture was derived from the prostate of a 23-year old organ donor; the BPH96-11 culture was derived from a 56-year old with benign prostatic hyperplasia (BPH). Two of these cell lines, DU 145 and PC-3, as well as the primary cultures, can synthesize 1,25(OH)₂D from its metabolic precursor 25(OH)D. The vitamin D metabolite 1,25(OH)₂D was also synthesized from 25(OH)D by cultured normal human keratinocytes. The results are shown in Table 1 below:

TABLE 1
Synthesis of 1,25(OH)₂D in the presence and
absence of the P450 cytochrome inhibitor, clotrimazole.

| | 1,25(OH)₂D Produced (pmol/mg protein/hr) | |
|---|---|---|
| CELL TYPE | P-450 Inhibitor Absent | P-450 Inhibitor Present |
| DU 145 | 0.31 ± 0.06 (n = 6) | undetectable |
| PC-3 | 0.07 ± 0.01 (n = 6) | undetectable |
| LNCaP | undetectable | undetectable |
| NP96-5 | 3.08 ± 1.56 (n = 3) | undetectable |
| BPH96-11 | 1.05 ± 0.31 (n = 3) | undetectable |
| Human keratinocytes | 2.1 ± 0.1 (n = 3) | undetectable |

n = number of replications, errors are S.E.M.

As summarized in Table 1, the DU 145 and PC-3 cell lines produced 0.31±0.06 and 0.07±0.01 pmol of 1,25(OH)₂D mg protein/hr, respectively. These cells exhibited 1α-OHase activity, as detected by thymus receptor binding assay according to well-known procedures. The production of 1,25(OH)₂D was completely inhibited in the presence of clotrimazole, a commonly known inhibitor of the cytochrome P450 system, of which 1α-OHase is a known component. Conversely, no measurable 1,25(OH)₂D was detected in LNCaP cells, which did not exhibit 1α-OHase activity.

Primary cultures of prostatic cells from two patients were also grown, NP96-5 (normal prostate) and BHP96-11 (BPH), and their enzyme activities determined in the presence of DPPD, and in the presence or absence of clotrimazole. The NP96-5 and BPH96-11 cultures produced 3.08±1.56 pmol/mg protein/hr and 1.05±0.31 pmol/mg protein/hr of 1,25(OH)₂D, respectively, in the presence of DPPD and in the absence of cytochrome P450 inhibitor. The 1α-OHase activity found in the two primary cultures of prostatic cells was comparable to that found in the normal human keratinocytes. As in the two cell lines, the production of 1,25(OH)₂D in the primary cultures of prostatic cells and keratinocytes was completely inhibited in the presence of clotrimazole.

The enzyme activity detected in the primary cultures of prostatic cells was further supported by high performance liquid chromatography analysis using a solvent system that specifically separates 1,25(OH)₂D from 10-oxo-19-nor-25(OH)D, another metabolite of 25(OH)D. This metabolite, which is present in significant quantity in kidney homogenates of rats and chickens, is known to co-migrate with 1,25(OH)₂D on normal phase HPLC with the n-hexane:isopropanol (9:1) solvent system. Therefore, to ensure that 1,25(OH)₂D was separated from any 10-oxo-19-nor-25(OH)D present, the methylene chloride:isopropanol (19:1) normal phase solvent system was used. In addition, 1α-OHase activity was determined by using a radioactive metabolite, [³H]-25(OH)D, as substrate.

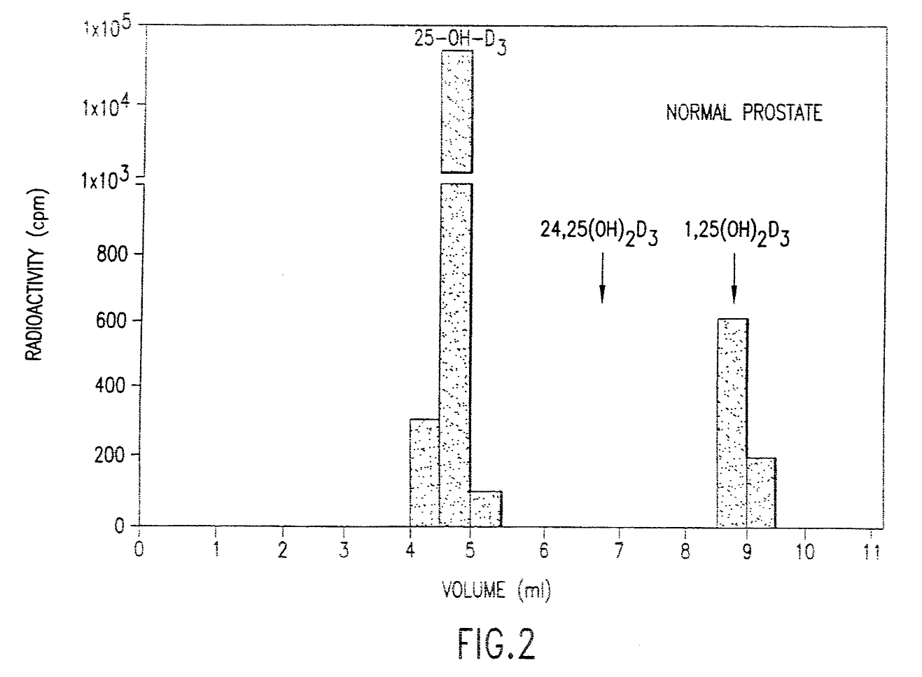

Typical HPLC chromatograms of the two primary prostate cell cultures in the presence and absence of clotrimazole are illustrated in FIGS. 2-5. Second passage of normal human prostate cells incubated with nonradioactive 25(OH)D₃ (50 nM), [³H]-25(OH)D₃ (0.91 μCi/nmol) and 1,2-dianilinoethane (DPPD) (10 μM) at 37° C. for two hours in the absence of clotrimazole (20 μM) is shown in FIG. 2. The active metabolite, 1,25(OH)₂D, was synthesized in detectable levels (about 600 cpm).

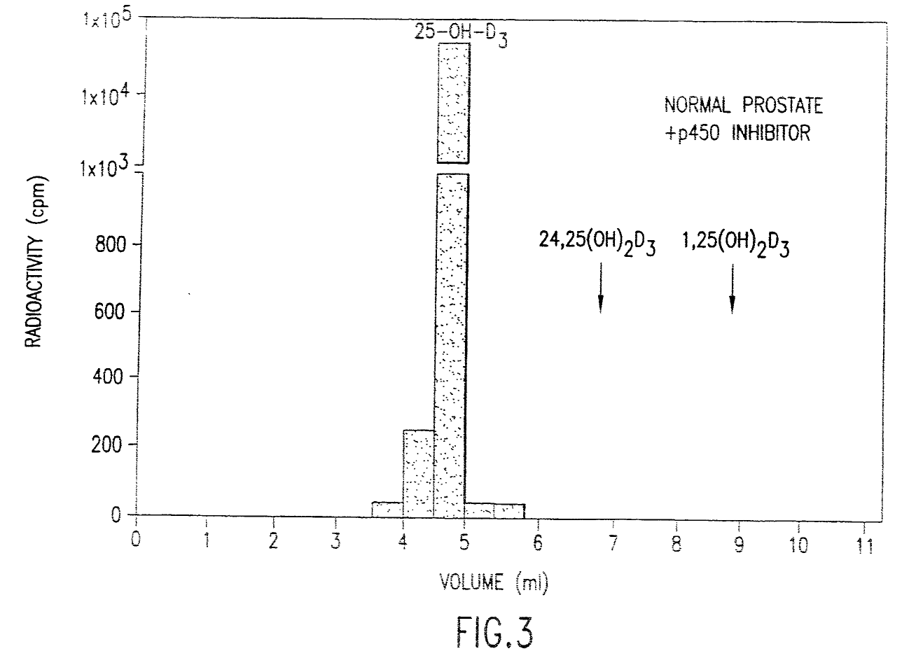

Second passage of normal human prostate cells were also incubated with nonradioactive 25(OH)D₃ (50 nM), [³H]-25(OH)D₃ (0.91 μCi/nmol) and DPPD (10 μM) at 37° C. for two hours in the presence of clotrimazole (20 μM) in the presence of the cytochrome P450 inhibitor (clotrimazole). FIG. 3 shows that 1,25(OH)₂D synthesis was significantly inhibited, whereby the active metabolite was undetectable.

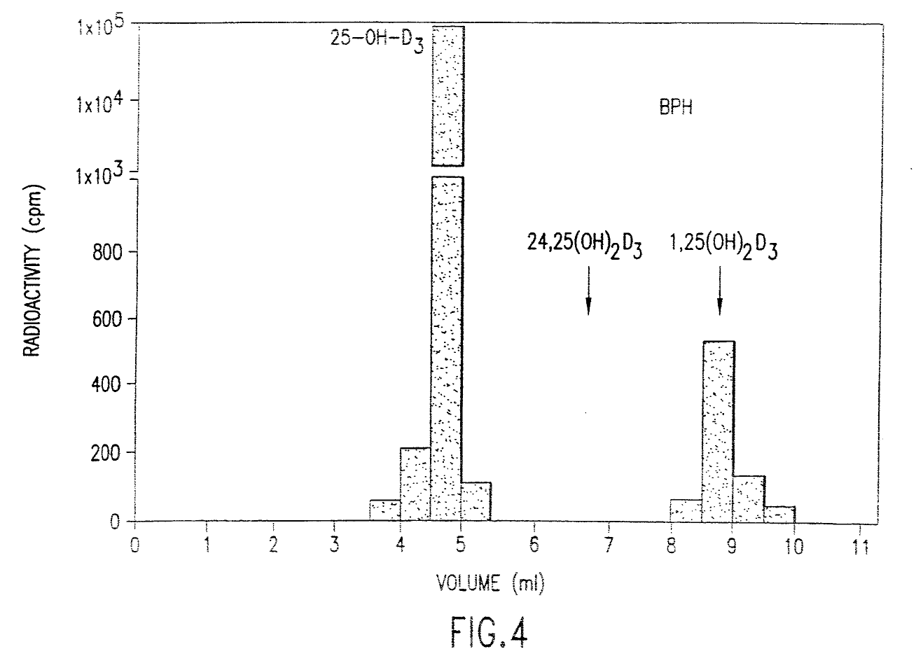

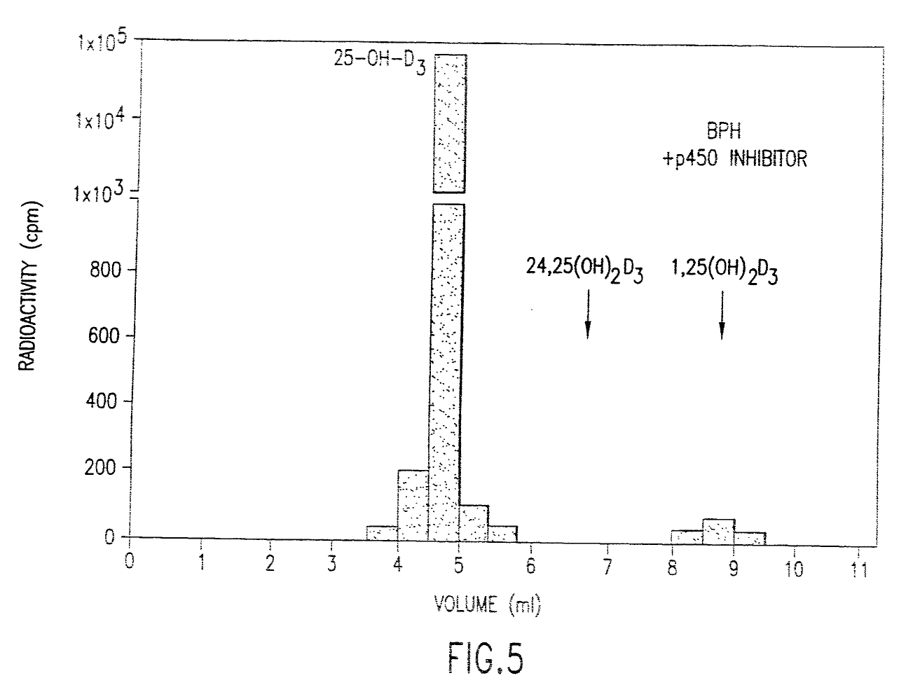

Second passage of prostate cells derived from BPH cultures were incubated with nonradioactive 25(OH)D₃ (50 nM), [³H]-25(OH)D₃ (0.91 μCi/nmol) and DPPD (10 μM) at 37° C. for two hours in the absence of clotrimazole (20 μM). Without cytochrome P450 inhibitor, active metabolite was detectable, producing product which generated about 500 cpm. See FIG. 4. By contrast, second passage of prostate cells derived from BPH cultures incubated with nonradioactive 25(OH)D₃ (50 nM), [³H-25(OH)D₃ (0.91 μCi/nmol) and DPPD (10 μM) at 37° C. for two hours in the presence of clotrimazole (20 μM), showed significantly reduced levels of the 1,25(OH)₂D metabolite, measuring less than about 200 cpm (FIG. 5).

Both primary cultures of non-cancerous prostate cells produced 1,25(OH)₂D at levels 10-40 fold higher than the cell lines. Very high levels of 1,25(OH)₂D were observed from the NP96-5 culture derived from the 23 year old organ donor. The quantities of 1,25(OH)₂D produced by the two primary non-cancerous human prostate cells, 3.08±1.56 and 1.05±0.31 pmol/mg protein/hr (normal and BPH, respectively), are comparable to those produced in cultured human keratinocytes (2.1±0.1 pmol/mg protein/hr), and to the HEP 62 hepatoma cell line (2.3 pmol/mg protein/hr), and human T-lymphotrophic virus-transformed lymphocytes (1.6 pmol/mg protein/hr). These values are at least 10-fold higher than those reported in other extra-renal sites, such as human bone cells (0.068 pmol/mg protein/hr).

After incubation with 25(OH)D₃, DU 145 and PC-3 prostate cancer cell lines also produced detectable levels of 1,25(OH)₂D₃. The 100% inhibition of 1,25(OH)₂D production by the specific cytochrome P450 inhibitor, clotrimazole, shows that a cytochrome P450-dependent 1α-OHase is present in these prostate cells. The addition of the P-450 inhibitor inhibited substantially all the conversion of [³H]-25(OH)D₃ to [³H]-1,25(OH)₂D₃ in these cells. Thus, DU 145 and PC-3 human prostate cancer cells, as well as two primary cultures derived from non-cancerous human prostates, possess 1α-OHase activity and are capable of converting the major circulating metabolite of vitamin D₃, 25(OH)D₃, to the hormonally active vitamin D metabolite, 1,25(OH)₂D₃.

The enzyme activity found in the primary cultures of prostate cells is comparable to that found in the primary cultures of renal proximal tubular cells, 5.3-5.6 pmol/hr/mg protein. Since it is established that renal 1α-OHase is the major enzyme responsible for maintaining the circulating concentration of 1,25(OH)₂D under normal physiological conditions, the amount of 1,25(OH)₂D produced by prostatic cells can be physiologically significant, especially for the micro environment of prostatic cells. Levels of 1,25(OH)₂D as low as 10⁻¹¹ M can significantly inhibit invasiveness of human prostate cancer cells through an artificial basement membrane composed of human amnions (Schwartz, G. G., et al., Cancer Epidemiol. Biomark. Prev. 6:727-732 (1997)).

No detectable 1,25(OH)₂D₃ was produced by LNCaP cells. 24,25-hydroxylase activity has also been reported to be low or undetectable in LNCaP. See Miller, G. J., Cancer Res. 52:515-520 (1992); Skowronski, R. J., Endocr. Rev. 132:1952-1960 (1993).

The unique discovery that 1,25(OH)₂D can be synthesized from 25(OH)D by prostatic cells provides the nexus for using vitamin D metabolites which are comparatively less toxic than high doses of vitamin D or certain of other metabolites, including 1,25(OH)₂D, in cancer treatment. The method of the subject invention comprising administering to a patient a vitamin D metabolite which can be converted to 1,25(OH)₂D by the enzyme 1α-OHase is preferably used in chemoprevention of prostate cancer. However, it would be understood by those of ordinary skill in the art that, in view of the principles and procedures described herein, cancer cells in other organs or tissues which are capable of converting vitamin D metabolite to 1,25(OH)₂D can respond to the antiproliferative, anti-invasive, antimetastatic, or prodifferentiating properties of 1,25(OH)₂D. Accordingly, it would be understood that the subject invention can be applicable to other cancer cells, including colon cancer cells, breast cancer cells, leukemia cells, skin cancer cells, lung cancer cells, and lymphoma cancer cells.

The product synthesized from the administered metabolite, 1,25(OH)₂D, exerts anti-proliferative and pro-differentiating effects on normal and cancerous prostate cells. Moreover, physiological levels of 1,25(OH)₂D significantly inhibit the invasiveness of DU 145 cells through an artificial basement membrane, as correlated with a decrease in the secreted levels of type IV collagenase (MMP-2 and MMP-9). It has further been shown that 1,25(OH)₂D exhibits antimetastatic effects on prostate cancer cells in vivo. However, administration of 1,25(OH)₂D has particular disadvantages due to its causative effect in producing hypercalcemia. Thus, 1,25(OH)₂D is not considered by those in the art to be optimal for safe use as a chemopreventive agent because of this risk of hypercalcemia. Our present findings indicate that by increasing the available substrate, e.g., administering or supplementing 25(OH)D to a patient, local synthesis of 1,25(OH)₂D by prostatic cells can be achieved, making increased levels of the active metabolite 1,25(OH)₂D available to the prostatic cells, and thereby reducing proliferation, invasiveness, or metastatic activity of cancerous prostate cells, and promoting their differentiation.

As described herein, 25(OH)D and analogs and derivatives thereof can be useful in several clinical settings, e.g., in slowing or preventing the need for prostatectomy or radiation therapy in men on “watchful waiting,” and/or slowing or preventing disease recurrence in men with minimal residual disease.

The vitamin D metabolite 25(OH)D is advantageously stored in smaller proportions in body fat as compared to vitamin D or other more fat-soluble metabolites or analogs of vitamin D. Moreover, 25(OH)D bypasses hepatic 25-hydroxylation. Therefore, its onset and offset of action are faster than vitamin D. In addition, 25(OH)D can be effective when there is compromise of the hepatic 25-hydroxylation of vitamin D₂ or D₃, as in primary biliary cirrhosis or certain cases of neonatal hypocalcemia. Further, 25(OH)D may have some intrinsic agonist activity that may retain some level of efficacy in the absence of the renal 1α-OHase system.

The subject method, including the preferred method of administering 25(OH)D or an analog, derivative, salt, or functional equivalent thereof, would not be limited to treatment of prostatic cells. Those cells having 1α-OHase can also convert 25(OH)D to 1,25(OH)₂D, thereby increasing local cellular levels of the 1,25(OH)₂D end product. Those cells which also have VDRs which bind 1,25(OH)₂D, or its functional equivalent, can respond to that compound and benefit from its antiproliferative, anti-invasive, antimetastatic, and prodifferentiating effects. For example, colon or breast cells can respond to 1,25(OH)₂D due to their possessing 1α-OHase and VDRs, either inherently or by use of genetic engineering techniques well-known in the art.

The dosage administration to a host in the above indications will be dependent upon the identity of the disease or condition, the type of patient involved, its age, weight, health, kind of concurrent treatment, if any, frequency of treatment, and therapeutic ratio.

The compounds of the subject invention can be formulated according to known methods for preparing pharmaceutically useful compositions. Formulations are described in detail in a number of sources which are well known and readily available to those skilled in the art. For example, Remington's Pharmaceutical Science by E. W. Martin describes formulations which can be used in connection with the subject invention. In general, the compositions of the subject invention will be formulated such that an effective amount of the bioactive compound(s) is combined with a suitable carrier in order to facilitate effective administration of the composition.

In accordance with the invention, pharmaceutical compositions comprising, as active ingredient, an effective amount of one or more of the subject compounds and one or more non-toxic, pharmaceutically acceptable carriers or diluents can be used by persons of ordinary skill in the art. In addition, the pharmaceutical composition can comprise one or more of the subject compounds, e.g., 25(OH)D, as a first active ingredient plus a second active ingredient, e.g., an anti-inflammatory, antimicrobial, antiproliferative, or antiviral compound, known in the art.

In accordance with this invention, pharmaceutically effective amounts of a known second active ingredient and the vitamin D metabolite, analog or derivative thereof useful in accordance with the subject invention are administered sequentially or concurrently to the patient. The most effective mode of administration and dosage regimen of subject compounds will depend upon the type of disease to be treated, the severity and course of that disease, previous therapy, the patient's health status, and response to vitamin D metabolites, analogs and derivatives, and the judgment of the treating physician. The subject compositions may be administered to the patient at one time or over a series of treatments.

Preferably, the subject compound and the second agent are administered sequentially to the patient, with the second agent being administered before, after, or both before and after treatment with the subject compound or composition. Sequential administration involves treatment with the second agent at least on the same day (within 24 hours) of treatment with the subject vitamin D metabolite and may involve continued treatment with the second agent on days that the subject compound is not administered. Conventional modes of administration and standard dosage regimens for the subject compound or a pharmaceutical composition comprising the subject compound may be used (see Gilman, A. G., et al. (eds.), The Pharmacological Basis of Therapeutics, pp. 697-713, 1482, 1489-91(1980); Physicians Desk Reference, 1986 Edition), similar to other compounds used for the treatments, or can be titrated for the individual patient.

According to one embodiment of this invention, the patient may receive concurrent treatments with compositions comprising at least one of the subject compounds, and a second active ingredient. For example, local, intra-tissue (or organ) injection of 25(OH)D or analog or derivative thereof is preferred but can be administered by subcutaneous injection, subcutaneous slow-release implant, intravenously, intraperitoneally, topically, intramuscularly, or orally.

The compositions used in these therapies can also be in a variety of forms. These include, for example, solid, semi-solid, and liquid dosage forms, such as tablets, pills, powders, liquid solutions or suspension, suppositories, injectable and infusible solutions. The preferred form depends on the intended mode of administration and therapeutic application. The compositions also preferably include conventional pharmaceutically acceptable carriers and adjuvants which are known to those of skill in the art. Preferably, the compositions of the invention are in the form of a unit dose and will usually be administered to the patient one or more times a day.

### Gene Therapy

Further, the subject invention can employ a gene therapy approach utilizing molecular biology techniques or procedures whereby a target cell or tissue is genetically altered to respond to the active vitamin D metabolite or analog and derivative thereof, e.g., 1,25(OH)₂D, by transgenically providing the target cell with a gene which encodes a protein or plurality of proteins which serve as vitamin D receptors (VDRs) that bind 1,25(OH)₂D. Alternatively, the target cells can be genetically engineered to encode and produce 1α-OHase, which is known to convert 25(OH)D to 1,25(OH)₂D. The genes of interest can be identified using standard probe and sequencing techniques, cloned or otherwise amplified, isolated and inserted into appropriate vectors for transfer into the target cells. Further, the target cells can be provided with promoter sequences which activate the appropriate gene sequence in the genome of the target cell to instruct the cell to produce the desired proteins or promoters.

The gene therapy approach of the present invention can be used as a method to treat or prevent cancer. As used herein, such cancers include, but are not limited to, Hodgkin's disease, non-Hodgkin's lymphomas, acute lymphocytic leukemia, multiple myeloma, breast carcinomas, ovarian carcinomas, lung carcinomas, Wilms' tumor, testicular carcinomas, soft-tissue sarcomas, chronic lymphocytic leukemia, primary macroglobulinemia, bladder carcinomas, chronic granulocytic leukemia, primary brain carcinomas, malignant melanoma, small-cell lung carcinomas, stomach carcinomas, colon carcinomas, malignant pancreatic insulinoma, malignant carcinoid carcinomas, malignant melanomas, choriocarcinomas, mycosis fungoides, head and neck carcinomas, osteogenic sarcoma, pancreatic carcinomas, acute granulocytic leukemia, hairy cell leukemia, rhabdomyosarcoma, Kaposi's sarcoma, genitourinary carcinomas, thyroid carcinomas, esophageal carcinomas, malignant hypercalcemia, renal cell carcinomas, endometrial carcinomas, polycythemia vera, essential thrombocytosis, adrenal cortex carcinomas, skin cancer, and prostatic carcinomas.

In this preferred embodiment of the invention, 1α-OHase DNA (e.g., the nucleic acid sequence depicted in SEQ ID NO. 1) can be incorporated into a polynucleotide construct suitable for introducing the nucleic acid molecule into cells of the animal to be treated, to form a transfection vector. The transfection vector is then introduced into selected target tissues of the cells of the animal in vivo using any of a variety of methods known to those skilled in the art. Alternatively, naked DNA may be transfected into the cells, with or without cationic lipids.

Techniques for the construction of transfection vectors containing 1α-OHase DNA are well-known in the art, and are generally described in “Working Toward Human Gene Therapy,” Chapter 28 in Recombinant DNA, 2nd Ed., Watson, J. D. et al. (eds.), Scientific American Books: New York (1992), pp.567-581, or Sambrook et al., Molecular Cloning: A Laboratory Manual, Cold Spring Harbor, N.Y. (1989).

Gene therapy approaches that may be used to deliver a VDR and/or 1α-OHase gene include injection of plasmid DNA (Horton, H. M., et al., Proc. Natl. Acad. Sci. USA 96(4):1553-1558 (1999)); transduction using adenoviral vectors (Waugh, J. M., et al., Proc. Natl. Acad. Sci. USA 96(3):1065-1070 (1999)); transduction using retrovial vectors (Axelrod, J. H., et al., Proc. Natl. Acad. Sci. USA 87:5173-5177 (1990); Drumm, M. L., et al., Cell 62:1227-1233 (1990); Krueger, G. G., et al., J. Invest. Dermatol. 112:233-239 (1999), Palmer, T. D., et al., Blood 73:438-445 (1989); and Rosenberg, S. A., et al., N. Eng J. Med. 323:570-578 (1990)); and gene transfer using liposomes (Mason, C. A. E., et al., Nature Medicine 5(2):176-182 (1999)). In addition, general methods for construction of gene therapy vectors and the introduction of such vectors into a mammal for therapeutic purposes may be obtained in the above-referenced publications, the disclosures of which are specifically incorporated herein by reference in their entirety. In one such general method, vectors containing 1α-OHase DNA of the present invention are directly introduced into the cells or tissues of the mammal to be treated, preferably by injection, inhalation, ingestion, topical application, or introduction into a mucous membrane via solution. Such an approach is generally referred to as “in vivo” gene therapy.

Alternatively, cells or tissues may be removed from the mammal to be treated and placed into culture according to methods that are well-known to one of ordinary skill in the art. Transfection vectors or naked DNA containing the 1α-OHase DNA may then be introduced into these cells or tissues by any of the methods described generally above for introducing isolated polynucleotides into a cell or tissue. After a sufficient amount of time to allow incorporation of the 1α-OHase DNA, the cells or tissues may then be re-inserted into the mammal to be treated. Since introduction of the 1α-OHase gene is performed outside of the body of the mammal, this approach is generally referred to as “ex vivo” gene therapy. See U.S. Pat. No. 5,399,346. Gene transfer through transfection of cells ex vivo can be performed by a variety of methods, including, for example, calcium phosphate precipitation, diethylaminoethyl dextran, electroporation, lipofection, or viral infection. Such methods are well known in the art (see, for example, Sambrook et al.).

The term “gene,” as used herein with reference to 1α-OHase, is intended to refer to a DNA sequence that encodes the 1α-OHase enzyme. Thus, “1α-OHase gene” or “gene expressing 1α-OHase” may refer to either a segment of genomic DNA encoding the 1α-OHase enzyme or a cDNA sequence encoding the 1α-OHase enzyme.

For both in vivo and ex vivo gene therapy, the 1α-OHase DNA of the invention may be operatively linked to the regulatory DNA sequence, or “promoter,” for human 1α-OHase (depicted in SEQ ID NO. 3) to form a genetic construct as described above. This construct, containing both the human 1α-OHase promoter and the 1α-OHase DNA, may be subcloned into a suitable vector such as a plasmid, adenovirus vector, retrovirus vector, or the like, and introduced into the animal to be treated in an in vivo gene therapy approach, or into the cells or tissues of the mammal in an ex vivo approach.

Alternatively, the 1α-OHase DNA of the invention may be operatively linked to a heterologous regulatory DNA sequence, or promoter, to form a genetic construct as described above. The heterologous regulatory sequence may be tissue specific. The vector containing the genetic construct is then directly introduced into the animal to be treated or into the cells or tissues of the animal, as described.

The term “operably linked”, as used herein, denotes a relationship between a regulatory region (typically a promoter element, but may include an enhancer element) and the gene, whereby the transcription of the gene is under the control of the regulatory region.

The term “heterologous” means a DNA sequence not found in the native genome. That is, two nucleic acid elements are said to be “heterologous” if the elements are derived from two different genes, or alternatively, two different species. Thus, “heterologous DNA regulatory sequence” indicates that the regulatory sequence is not naturally ligated to the DNA sequence for the 1α-OHase gene.

The term “promoter” is used according to its art-recognized meaning. It is intended to mean the DNA region, usually upstream to the coding sequence of a gene, which binds RNA polymerase and directs the enzyme to the correct transcriptional start site.

In general, a promoter may be functional in a variety of tissue types and in several different species of organisms, or its function may be restricted to a particular species and/or a particular tissue. Further, a promoter may be constitutively active, or it may be selectively activated by certain substances (e.g., a tissue-specific factor), under certain conditions (e.g., in the presence of an enhancer element, if present, in the genetic construct containing the promoter), or during certain developmental stages of the organism (e.g., active in fetus, silent in adult).

Promoters useful in the practice of the present invention are preferably “tissue-specific”—that is, they are capable of driving transcription of a gene in one tissue while remaining largely “silent” in other tissue types. Examples of tissue-specific promoters are provided in Table 2 below.

TABLE 2
Tissue Specific Promoters

| Gene | Species | Tissue Specificity | Ref. |
|---|---|---|---|
| alpha - Actin | rat | mu, he | 1 |
| alpha - Actin | rat | te, th, lu | 1 |
| Elastase - I | rat | Pa | 2 |
| alpha - Fetoprotein | mouse | ys, li | 3 |
| beta - Globin | human | ery | 4 |
| beta - Globin | rabbit | te, mu | 5 |
| beta - Globin | rabbit | ery | 6 |
| tau - Globin | human | ery | 7 |
| alpha - Globin | mouse | br | 8 |
| Growth hormone | human | pit | 9 |
| Immunoglobin - kappa | mouse | B | 10 |
| Immunoglobin - mu | mouse | B, T | 11 |
| Insulin | human | beta —cells | 12 |
| Myosin Light Chain-2 | rat | mu | 13 |
| Protamine 1 | mouse | te | 14 |
| alpha - A-crystallin | trans | lens | 15 |
| Prolactin | * | pit | 16 |
| Pro-opiomelanocortin | * | pit | 17 |
| BTSH | * | | 18 |
| MMTV | mouse | breast | 19 |
| Albumin | * | li | 20 |
| Keratin | * | skin | 21 |
| Osteonectin | * | bone | 22 |
| Prostate | * | prostate | 23 |
| Olfactory Marker Protein | * | neuron | 24 |
| Neuron Specific Enolase (NSE) | * | neuron | 25 |
| L-7 | * | neuron | 26 |
| Opsin | * | retina | 27 |
| Glial Fibrillary Acidic Protein | human | astrocytes (CNS) | 28 |
| (GFAP) | | | |
| Tyrosine hydroxylase (TH) | rat | catecholaminergic | 29 |
| | * | neurons | |
| | human | * | 30 |
| Amyloid precursor protein (APP) | human | neurons | 31 |
| | * | * | 32 |
| Dopamine beta - hydroxylase | human | noradrenergic | 33 |
| (DBH) | | neurons | |
| | * | adrenergic neurons | 34 |
| Myelin basic protein (MBP) | mouse | oligodendrocyte | 35 |
| Light neurofilament (NF-L) | rat | neurons | 36 |
| Tryptophan hydroxylase (TH) | human | serotonin/pineal | 37 |
| | | gland | |
| | mouse | * | 38 |
| Purkinje cell protein-2 (Pcp-2) | mouse | Purkinje | 39 |
| | | cells/cerebellum | |
| L7 | mouse | cerebellar Purkinje | 40 |
| | | retinal bipolar cells | |
| Type II sodium channel | rat | neuron | 41 |
| Choline acetyltransferase (ChAT) | human | cholinergic neurons | 42 |
| | rat | * | 43 |
| Neuron specific enolase (NSE) | rat | neurons | 44 |
| Aromatic L-amino acid | human | catecholaminergic/ | 45 |
| decarboxylase (AADC) | | 5-HT/D-type cells | |
| Protamine 1 (mP1) | mouse | spermatids | 46 |
| Proenkephalin | human | neuronal/ | 47 |
| | | spermatogenic | |
| | | epididymal cells | |
| | rat | * | 48 |
| reg (pancreatic stone protein) | human | colon and rectal | 49 |
| | | tumors, pancreas, | |
| | | kidney | |
| Parathyroid hormone-related | human | Liver and cecum | 50 |
| peptide (PTHrP) | | tumors, | |
| | | neurilemoma, | |
| | | kidney, pancreas, | |
| | | adrenal | |
| Stromelysin 3 | human | breast cancer | 51 |
| NSE (see nervous system) | * | small-cell lung | 52 |
| | | cancer, neurons | |
| AADC (see nervous system) | * | neurectodermal | 53 |
| | | tumors | |
| Albumin | * | hepatoma | 54 |
| c-erbB3 | * | breast cancer | |
| c-erbB4 | * | breast and gastric | |
| | | cancer | |
| Thyroglobulin | * | thyroid carcinoma | |
| alpha —fetoprotein | * | hepatoma | 55 |
| hemoglobin | * | erythrocytes | 56 |

Abbreviations:
br, brain;
B, lymphocytes;
mu, skeletal muscle;
he, cardiac muscle;
te, testis;
beta, beta cells;
th, thymus;
lu, lung;
Pa, exocrine pancreas;
ys, yolk sac;
li, liver;
ery, erythroid cells;
pit, pituitary and
lens, eye lens.

### References

(1) Shani, Mol. Cell. Biol., 6:2624 (1986); (2) Swift et al., Cell 38:639 (1984); (3) Krumlauf et al., Nature 319:224 (1985); (4) Townes et al., EMBO J. 4:1715 (1985); (5) Lacy et al., Cell 34:343 (1983); (6) Wagner et al., Proc. Natl. Acad. Sci. U.S.A., 78:6376 (1981); (7) Brinster et al., Nature 283:499 (1980); (8) Rusconi et al., in “The Impact of Gene Transfer Techniques in Eukaryotic Cell Biology,” ed. J. S. Schell et al., pp. 134-152, Berlin: Springer Verlag (1984); (9) Behringer et al., Genes Dev. 2:453 (1988); (10) Storb et al., Nature 310:238 (1984); (11) Grosschedl et al., Cell 38:647 (1984); (12) Selden et al., Nature 321:545 (1986); (13) Shani, Nature 314:283 (1985); (14) Peschon et al., Ann. N. York Acad, Sci., 564:186 (1989); (15) Breitman et al., Dev. 106:457 (1989); (16) Crenshaw et al., Genes and Development 3:959 (1989); (17) Tremblay et al., Proc. Natl. Acad. Sci. U.S.A. 85:8890 (1988); (18) Tatsumi et al., Nippon Rinsho 47:2213 (1989); (19) Muller et al., Cell 54:105 (1988); (20) Palmiter et al., Ann. Rev. Genet. 20:465 (1986); (21) Vassar et al., Proc. Natl. Acad. Sci. U.S.A. 86:8565 (1989); (22) McVey et al., J. Biol. Chem. 263:11 (1988); (23) Allison et al., Mol. Cell. Biol. 9:2254 (1989); (24) Danciger et al., Proc. Natl. Acad. Sci. U.S.A. 86:8565 (1989); (25) Forss-Petter et al., J. Neurosci. Res. 16:141 (1986); (26) Sutcliffe, Treads in Genetics 3:73 (1987); (27) Nathans et al., Proc. Natl. Acad. Sci. (U.S.A. 81:4851 (1984); (28) Brenner M., et al., J. Neurosci. 14:1030 (1994); (29) Kim, L. S., et al., J. Biol. Chem 268:15689 (1993); (30) Kaneda, N., et al., Neuron 6:583 (1991); (31) Salbaum, J. M., et al., EMBO J. 7:2807 (1988); (32) Wirak, D. O., et al., EMBO J. 10:289 (1990); (33) Mercer E. H., et al., Neuron 7:703 (1991); (34) Hcyle, G. W., et al., J. Neurosci. 14:2455 (1994): (35) Miura, M., et al., Gene 75:31 (1989); (36) Reeben, M., et al., J. Neurosci. Res. 40:177 (1995); (37) Boularand, S., et al., J. Biol. Chem 270:3757 (1995); (38) Stoll, J. and Goldman, D., J. Neurosci. Res. 28:457 (1991); (39) Vandaele, S., et al., Genes & Dev. 5:1136 (1991); (40) Oberdick, J., et al., Science 248:223 (1990); (41) Maue, R. A., et al., Neuron 4:223 (1990); (42) Hersh, L. B., et al., J. Neurochem. 61:306 (1993); (43) Ibanex, C. F. and Persson, H., Eur. J. Neurosci. 3:1309 (1991); (44) Forss-Petter, S., et al., Neuron 5:187 (1990); (45) Thai, A. L. V., et al., Mol. Brain Res. 17:227 (1993); (46) Peschon, J. J., et al., Proc. Natl. Acad. Sci. U.S.A. 84:5316 (1987); (47) Borsook, D., et al., Mol. Endocrinol. 6:1502 (1992); (48) Joshi, J. and Sabol, S. L., Mol. Endocrinol. 5:1069 (1991); (49) Watanabe, T., et al., J. Biol. Chem. 265:7432 (1990); (50) Campos, R. V., et al., Mol. Rnfovtinol. 6:1642 (1992); (51) Basset, P., et al., Nature 348: 699 (1990); (52) Bombardieri, E. et al., Eur. J. Cancer 31A:184 (1995); Koh, T. et al., Int. J. Cancer 60:843 (1995); (53) Thai, A. L. V., et al., Mol. Brain Res. 17:227 (1993); (54) Huber, B. E., PNAS 88:8099 (1991); (55) Zuibel, I., et al., J. Cell Physiol. 162:36 (1995); (56) Watanabe, T., et al., J. Biol. Chem. 265:7432 (1990).

For additional examples of tissue-specific promoters, see U.S. Pat. Nos. 5,834,306 and 5,416,027, and references cited therein.

In addition to a promoter, the genetic construct may also contain other genetic control elements, such as enhancers, repressible sequences, and silencers, which may be used to regulate replication of the vector in the target cell. The only requirement is that the genetic element be activated, derepressed, enhanced, or otherwise genetically regulated by factors in the host cell and, with respect to methods of treatment, not in the non-target cell.

An “element,” when used in the context of nucleic acid constructs, refers to a region of the construct or a nucleic acid fragment having a defined function. For example, a enhancer element, as used herein, is a region of DNA that, when associated with the 1α-OHase gene operably linked to a promoter, enhances the transcription of that gene.

The term “enhancer” is used according to its art-recognized meaning. It is intended to mean a sequence found in eukaryotes which can increase transcription from a gene when located (in either orientation) up to several kilobases from the gene being studied. These sequences usually act as enhancers when on the 5′ side (upstream) of the gene in question. However, some enhancers are active when placed on the 3′ side (downstream) of the gene. In some cases, enhancer elements can activate transcription from a gene with no (known) promoter.

Preferred enhancers include the DF3 breast cancer-specific enhancer and enhancers from viruses and the steroid receptor family. Other preferred transcriptional regulatory sequences include NF1, SP1, AP1, and FOS/JUN.

Any of a variety of methods known to those skilled in the art may be used to introduce transfection vectors of the present invention into selected target tissue cells. Such methods include, for example, viral-mediated gene transfer using retroviruses, adeno-associated virus (AAV), herpes virus, vaccinia virus, or RNA viruses (e.g., Grunhaus and Horowitz, Semin. Virol. 3:237-252 (1992); Herz and Gerard, Proc. Nat. Acad. Sci. USA 90:2812-2816 (1993); and Rosenfeld et al., Cell 68:143-155 (1992)); liposome-mediated gene transfer (Morishita et al., J. Clin. Invest. 91:2580 (1993); Felgner et al., U.S. Pat. Nos. 5,703,055 (1997) and 5,858,784 (1999)); injection of naked DNA directly into a target tissue (e.g., Felgner et al., U.S. Pat. No. 5,589,466 (1996); Wolff et al., U.S. Pat. No. 5,693,622 (1997)); and receptor-mediated gene transfer (Wu and Wu, Biochemistry 27:887-892 (1988); Wagner et al., PNAS USA 87:3410-3414 (1990); Curiel et al., U.S. Pat. No. 5,547,932 (1996); and Beug et al., U.S. Pat. No. 5,354,844 (1994)).

In any of these methods, where a vector may be targeted to selectively transfect a specific population of cells, it will be understood that in addition to local administration (such as may be achieved by injection into the target tissue), the vector may be administered systemically (e.g., intravenously) in a biologically-compatible solution or pharmaceutically acceptable delivery vehicle. Vector constructs administered in this way may selectively infect the target tissue. According to the present invention, the presence of a target tissue-specific promoter on the construct provides an independent means of restricting expression of the therapeutic gene.

The gene therapy embodiment of the invention provides a method of treating or preventing a number of diseases and disorders. For example, this embodiment can be used to treat or prevent cancer in cells that express the vitamin D receptor (VDR). A polynucleotide construct containing the gene coding for 1α-OHase is introduced into cancerous cells, where the gene is subsequently expressed, producing 1α-OHase in an amount effective to inhibit cell proliferation. A gene encoding the VDR (for example, the nucleotide sequence depicted in SEQ ID NO. 4) can also be co-administered with the 1α-OHase gene. In this embodiment of the invention, the VDR gene is subcloned into either the same polynucleotide construct as the 1α-OHase gene or into a separate polynucleotide construct, as described above. Both genes are then introduced into the target cell or tissue, where they are subsequently expressed, producing 1α-OHase and VDR in amounts effective to treat or prevent the condition.

Gene therapy with the gene expressing 1α-OHase and, optionally, the gene expressing the VDR gene may be practiced substantially simultaneously with administration of vitamin D or analog or derivative thereof. Thus, vitamin D or analog or derivative thereof may be administered to the animal before, during or after receiving gene therapy.

Specifically, the invention can be used to treat or prevent prostate cancer, breast cancer, skin cancer, colon cancer, leukemias, lymphomas, and lung cancer.

The invention also provides a method of treating or preventing any disease in which 1,25(OH)₂D₃ can influence cell growth and maturation, including pre-cancers such as actinic keratoses, and non-cancerous hyperproliferative disorders such as psoriasis and ichthyosis. In this embodiment of the invention, a polynucleotide construct containing the gene coding for 1α-OHase is introduced into cells in which 1,25(OH)₂D₃ can influence cell growth and maturation. The 1α-OHase gene is subsequently expressed, producing 1α-OHase in an amount effective to inhibit cell proliferation.

The invention can therefore be used in the treatment of actinic keratoses, which is a pre-skin cancer. Use of the invention would effect an increase of the amount of 1,25(OH)₂D₃ (or analog or derivative thereof) within the cell, decreasing proliferative activity and inducing the cells to differentiate into normal morphology.

Further, the invention can also be used to treat hyperproliferative disorders of the skin such as psoriasis. This gene therapy approach is a reasonable alternative treatment for psoriasis, whereby the cells would produce or boost their production of 1,25(OH)₂D₃ (or analog or derivative thereof) which, in turn, would interact with the VDR and decrease the proliferative activity making the skin cells normal. This treatment can also be applied to other hyperproliferative skin disorders such as ichthyosis.

The invention can also be used to treat or prevent disorders in calcium and bone metabolism. Again, a polynucleotide construct containing the gene coding for 1α-OHase can be introduced into a group of cells of the animal, where it is subsequently expressed, enabling these cell to produce 1,25(OH)₂D₃ (or analog or derivative thereof) in a manner similar to the kidney. Thus, the invention may be used to prevent bone disease associated with kidney failure and treat vitamin D-dependent rickets type I, X-linked hypophosphatemic rickets, vitamin D-dependent rickets type II, and osteoporosis, as well as such disorders in calcium and bone metabolism as low blood serum calcium due to hypoparathyroidism.

A further embodiment of the invention provides a method of treating or preventing any disease or condition mentioned above, including cancer, by administering the gene encoding the VDR as well as the gene coding for 1α-OHase to a target cell or tissue of an animal. Both genes may be subcloned into either the same or different polynucleotide constructs, as described above, which is introduced into the target cell or tissue. Both the 1α-OHase gene and the VDR gene are subsequently expressed, producing 1α-OHase and VDR in amounts effective to treat or prevent the condition.

This particular embodiment may be used with cells that either express or do not already express the VDR. In the former case (in which the target cell already expresses the VDR), the cell will produce additional VDR which will interact with the increased cellular concentrations of 1,25(OH)₂D (or analog or derivative thereof). In the latter case (in which the cells do not already express VDR), the cell will become responsive to 1,25(OH)₂D (or analog or derivative thereof). For example, a cancer cell that had either a defective or absent VDR could be treated according to this embodiment of the invention. The treated cell would then produce VDR which would, in turn, interact with the 1,25(OH)₂D (or analog or derivative thereof) that was produced from the 1α-OHase. The interaction leads to a biologic response, including inhibiting proliferation and inducing terminal differentiation, thereby treating cancer, pre-cancer, non-cancerous hyperproliferative disorders, enhancing wound healing, and treating and preventing hair loss.

The invention also provides a method of treating benign prostatic hyperplasia in an animal, by introducing into cells exhibiting benign prostatic hyperplasia a polynucleotide construct containing a gene coding for 1α-OHase. The 1α-OHase gene is subsequently expressed enabling the cells to produce 1,25(OH)₂D (or an analog thereof) in an amount effective to treat the benign prostatic hyperplasia. In this embodiment, a gene encoding the VDR can also be co-administered with the 1α-OHase gene, as described above. The gene expressing 1α-OHase and the gene expressing the VDR can be on the same DNA construct or on separate DNA constructs.

The invention also provides a method of treating autoimmune diseases in an animal, by introducing into any cells of the animal that would produce a beneficial result a polynucleotide construct containing a gene coding for 1α-OHase. The 1α-OHase gene is subsequently expressed, enabling the cells to produce 1,25(OH)₂D (or an analog thereof) in an amount effective to treat the autoimmune disease. In this embodiment, a gene encoding the VDR can also be co-administered with the 1α-OHase gene, as described above. The gene expressing 1α-OHase and the gene expressing the VDR can be on the same DNA construct or on separate DNA constructs.

The invention can be used to treat autoimmune diseases such as diabetes mellitus type 1, multiple sclerosis, rheumatoid arthritis, or psoriatic arthritis. In this embodiment, for example, diabetes mellitus type 1 can be treated by introduction of a polynucleotide construct(s) containing the 1α-OHase gene (and optionally the VDR gene) into any cell of the animal that would produce a beneficial result, particularly into pancreatic cells, and more particularly pancreatic cells secreting insulin, such as cells of the Islets of Langerhans. For use of the invention in the treatment of multiple sclerosis, the polynucleotide construct(s) containing the 1α-OHase gene (and optionally the VDR gene) can be introduced particularly into any neural cells that would produce a beneficial result. Similarly, for use of the invention in the treatment of rheumatoid arthritis or psoriatic arthritis, the polynucleotide construct(s) can be introduced particularly into any connective tissue cells that would produce a beneficial result, or in or around the joints of the animal affected by either of these diseases.

Further embodiments of the invention include isolated nucleic acid molecules that are at least 90% identical, and more preferably at least 95%, 97%, 98% or 99% identical to the above-described isolated nucleic acid molecules of the present invention. In particular, the invention is directed to isolated nucleic acid molecules at least 90%, 95%, 97%, 98%, or 99% identical to the nucleotide sequences depicted in SEQ ID NO. 1 and SEQ ID NO. 3. The “% identity” between two nucleic acid sequences can be determined using the “fastA” computer algorithm (Pearson, W. R. & Lipman, D. J., Proc. Natl. Acad. Sci. USA 85:2444 (1988)) with the default parameters.

The term “isolated polynucleotide molecule” is intended to refer to a nucleic acid molecule which has been removed from its native environment. For example, recombinant DNA molecules contained in a vector are considered isolated for purposes of the invention as are recombinant DNA molecules maintained in heterologous host cells or purified (partially or substantially) DNA molecules in solution. Isolated polynucleotide molecules also include such compounds produced synthetically.

The invention is further related to nucleic acid molecules capable of hybridizing to a nucleic acid molecule having a sequence complementary to or hybridizing directly to one of the nucleic acid sequences shown in SEQ ID NO. 1 and SEQ ID NO. 3 under stringent conditions. By “stringent conditions” is intended overnight incubation at 42° C. in a solution comprising: 50% formamide, 5×SSC (750 mM NaCl, 15 mM trisodium citrate), 50 mM sodium phosphate (pH 7.6), 5× Denhardt's solution, 10% dextran sulfate, and 20 μg/ml denatured, sheared salmon sperm DNA (ssDNA), followed by washing the filters in 0.1×SSC at about 65° C.

Following are examples which illustrate procedures for practicing the invention. These examples should not be construed as limiting. All percentages are by weight and all solvent mixture proportions are by volume unless otherwise noted.

### EXAMPLE 1

### Materials and Methods Used in Cell Culture Synthesis of 1,25(OH)₂D from 25(OH)D by Cell Cultures

(A) Cell Lines. DU 145, PC-3, and LNCaP cell lines were obtained from the American Type Culture Collection, Rockville, Md. All cell lines were tested and found to be free of Mycoplasma contamination.

(B) Culture Conditions. Prostate cancer cell lines were cultured following several passages in vitro. Cells were routinely cultured in complete medium (RPMI 1640 medium supplemented with fetal bovine serum (10%., HyClone Labs, Logan Utah) and gentamicin (10 μg/ml, Life Technologies, Inc., Gaithersburg, Md.)). Cells were seeded in 35 mm culture dishes (Corning-Costar, Cambridge, Mass.) at 1×10⁵ cells/dish in complete medium. Medium was changed to a serum-free medium (RPMI 1640 containing insulin (10 μg/ml), transferrin ( 10 μg/ml) and selenous acid (1 ng/ml)) 24 hours before adding 25(OH)D₃.

Primary cultures of human prostatic epithelial cells were established and characterized as described by Lokeshwar et al., Cancer Res. 53:4493-4498 (1993). Prostatic epithelial cells cultured in a serum-free defined medium (Mammary Epithelial Growth Medium (MEGM), Clontech, San Diego, Calif.) express luminal epithelium specific cytokeratins (cytokeratins 8 and 18) as detected immunohistochemically using an anti-cytokeratin antibody, CAM 5.2 (Becton-Dickinson, Mountain View, Calif.). The serum-free medium contains MCDF170 supplemented with Epidermal Growth Factor, 25 ng/ml, hydrocortisone, 0.5 μg/ml, ethanolamine, 1×10⁻⁴, insulin, 5 μg/ml, transferrin, 5 μg/ml, and whole bovine pituitary extract, 70 μg/ml.

Prostatic cells used were at their first passage in vitro and were cultured in the serum-free RPMI medium during incubation with vitamin D metabolites. Two primary cultures were investigated: NP96-5, cultured from the histologically normal prostate of a 23 year old Caucasian organ donor, and BPH96-11, cultured from an open prostatectomy specimen of a 56 year old Caucasian with BPH. Histological examination of adjacent tissue sections taken from specimens used for cultures confirmed their identity as normal or BPH cultures.

(C) Keratinocyte Culture. Because 1α-OHase activity has been well-established in keratinocytes, cultured human keratinocytes were used for comparison to the prostatic cultures. Keratinocytes were grown in culture following a modification of the method of Rheinwald and Green, Cell 6:331-344 (1975). Briefly, keratinocytes were obtained from neonatal foreskin after trypsinization at 4° C. Keratinocytes were plated and grown on lethally irradiated 3T3 fibroblast feeder cells in a serum-free basal medium containing 0.15 mM calcium and supplemented with growth factors including bovine pituitary extract (3 μg/ml), EGF (25 ng/ml), insulin (5 μg/ml) and prostaglandin E₁ (50 ng/ml). To enhance the plating efficiency, cholera toxin (0.1 μg/ml) and hydrocortisone (200 ng/ml) were added into the medium during the initial plating of the primary culture and the subsequent subcultures. Cells were fed and maintained without cholera toxin and hydrocortisone, and used for enzyme assay.

(D) 25-hydroxyvitamin D-1α-hydroxylase (1α-OHase) assay. 1α-OHase activity was determined in monolayer cultures of the cell lines and primary cultures described herein. The assays were performed in the presence of 25(OH)D₃ (50 nM) as the enzyme substrate and DPPD (Sigma-Aldrich, Allentown, Pa.), an antioxidant and a known inhibitor of free-radical generated 1,25(OH)₂D. Assays were also performed in the presence and absence of the cytochrome P450 inhibitor, clotrimazole (20 μM) (Sigma, St. Louis, Mo.). After two hours of incubation at 37° C., cultures were placed on ice and media were removed. Immediately afterward, 1 ml methanol was added to extract 25(OH)D₃ and 1,25(OH)₂D₃. A 10 μl aliquot of [³U]-1,25(OH)₂D₃ containing 1,000 cpm radioactivity in ethanol was also added to each well for calculating the recovery. After extraction at room temperature for fifteen minutes, the methanol extract was transferred to a glass test tube and the cells were washed with an additional 0.5 ml methanol. The extract and wash were combined, dried down with a stream of nitrogen, and redissolved in 1 ml acetonitrile followed by the addition of 1 ml 0.4M K₂HPO₄, pH 10.0. The mixture was then applied to a C-18-OH reversed phase cartridge. The fraction containing 1,25(OH)₂D was then dried down under a stream of nitrogen and reconstituted in 200 μl ethanol. Two 40 μl aliquots were taken for 1,25(OH)₂D analysis by thymus receptor binding assay as described by Chen, et al., J Nutr. Biochem. 1:320-327 (1990). The 1α-OHase activity was also determined in the primary cultures of prostatic cells by using 20 μg non-radioactive 25(OH)D₃ and 0.91 μCi of [³H]-25(OH)D₃ instead of only nonradioactive 25(OH)D₃ as a substrate. The incubation medium, time, temperature, extraction procedure, and C-18-OH cartridge chromatography were the same as described for the thymus receptor method, except that the fraction eluted from C-18-OH cartridge with 10% methylene chlorine in hexane (25(OH)D fraction) and with 6% isopropanol in n-hexane (1,25(OH)₂D and 24,25(OH)₂D fraction) was dried down under nitrogen and redissolved in methylene chloride: isopropanol (19:1) for high performance liquid chromatographic analysis as described below.

(E) High Performance Liquid Chromatography (HPLC). A 30 μl aliquot was mixed with 10 μl each of standard nonradioactive 25(OH)D and 1,25(OH)₂D and was applied to an Econosphere silica column (5 μ particle size 250 mm×4.6 mm) with a flow rate of 0.5 ml/min using methylene chloride: isopropanol (19:1) solvent system as the mobile phase. Thirty fractions were collected at one minute intervals from each HPLC. Fractions were allowed to evaporate by air to dryness, followed by adding scintillation fluid and counting with a beta counter. The retention volume for 25(OH)D₃, 24,25(OH)₂D₃ and 1,25(OH)₂D₃ was calibrated by applying standard 25(OH)D₃, 24,25(OH)₂D₃ and 1,25(OH)₂D₃ to the HPLC column prior, during, and after unknown sample application. The protein concentration in each 35 mm dish was determined by standard procedures. The enzyme activity was expressed as pmol 1,25(OH)₂D₃/mg protein/hr.

(F) Keratinocyte transfection. Keratinocytes were maintained in MCDB-153 medium. Cells in 24-well dishes at 50%-60% confluence were transfected with 1 mg/ml of 1α-OHase cDNA which had been subcloned into pCR3.1 vector (INVITROGEN). Empty vector was used as a control. For each transfection, 1.0 μg was diluted in 100 μl of medium containing 6-8 μl Plus Reagent. This mixture was incubated for 15 minutes at room temperature. LIPOFECTAMINE (4-6 μl) was added, and the DNA-LIPOFECTAMINE mixture was incubated at room temperature for an additional 15 minutes. Cells were incubated in the DNA-LIPOFECTAMINE mixture for 3 hours, after which the medium was changed. Cells were then treated with 25(OH)D₃ at different concentrations (1 nM, 10 nM, 100 nM) for 18 hours. [³H]thymidine was added to the cultures and [³H]thymidine incorporation into the DNA (interpreted as a measure of the DNA synthetic activity, i.e. proliferation of the cells) was measured as previously described (Chen, T. C., et al., J Nutr. Biochem. 4:49-57 (1993)).

### EXAMPLE 2

### Cloning, Sequencing, and Expression of Human Keratinocyte 1α-OHase cDNA

1α-OHase cDNA was cloned from human keratinocytes using reverse transcription-PCR (RT-PCR). The 2,150 base pair 1α-OHase cDNA (which is a part of SEQ ID NO. 1) contains a 1,527 bp open reading frame, predicted to encode protein of 508 amino acids (SEQ ID NO. 2). This cDNA was directly subcloned into the pCR3.1-Uni (INVITROGEN) mammalian cell expression vector. DNA sequence analysis showed the cDNA in keratinocytes was 100% identical to the renal 1α-OHase. When this construct was transfected in COS-1 cells, which cannot synthesize 1,25(OH)₂D₃, 1α-OHase activity was demonstrated by a high performance liquid chromatography (HPLC) which separated [³H]-1,25(OH)₂D₃ produced from the enzyme substrate, [³H]-25(OH)D₃.

### EXAMPLE 3

### Cloning and Partial Characterization of the Human 1α-OHase Gene

A human genomic DNA library (CLONTECH) was screened by plaque hybridization using a 700 bp fragment from the 5′ region of 1α-OHase cDNA (SEQ ID NO. 1) as a probe. Four positive clones were identified. Restriction enzymes were used to digest the four genomic clones which were designated P1, P2, P3, P4 respectively. (BamH I, Sac-I) P1 was 13 kb, and P2 was 7.5 kb. These two clones included some portion of the 5′ region as identified by PCR. The P1 clone was digested with Sac-I and generated four fragments of 5.5 kb, 3.5 kb, 3.0 kb and 0.7 kb in length. These four fragments were first subcloned into the pGem7 cloning vector, and southern blot analysis was applied to identify the subclone containing the 5′-flanking region. Sequence analysis using T7 and Sp6 promoter primers mapped the fragment order. The 5.5 kb fragment was found to encode intron 8 to exon 9. The 0.7 kb fragment encoded exon 6 to intron 8, the 3.5 kb encoded exon 6 to the 5′ flanking region, and the 3.0 kb subclone could not be hybridized with any probes made from 1α-OHase cDNA.

### EXAMPLE 4

### Proliferative Activity as a Function of 1,25(OH)₂D of Keratinocytes Transfected with the Human1α-OHase Gene

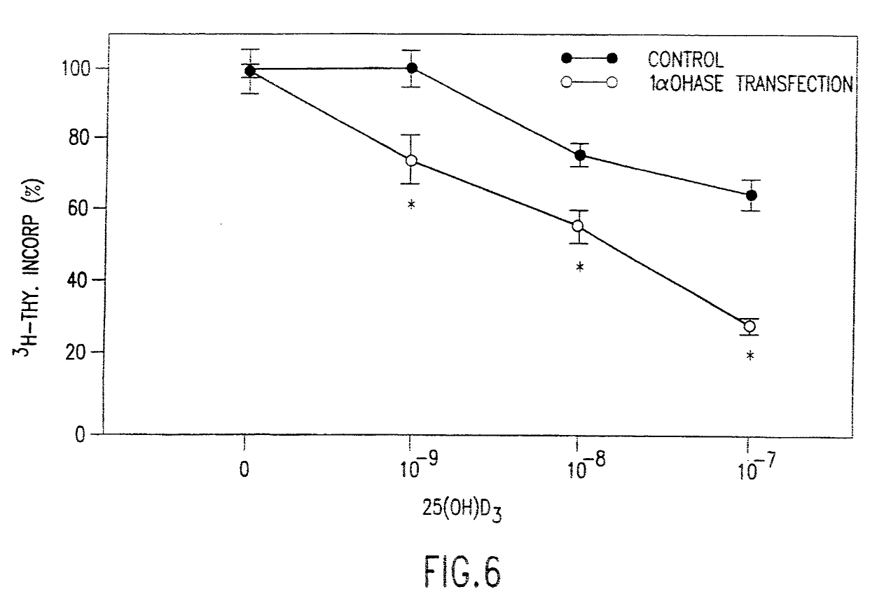

To test the ability of a cell to produce 1,25(OH)₂D₃ after transfection with 1α-OHase, a polynucleotide construct containing the 1α-OHase cloned gene (SEQ ID NO. 1) was constructed as outlined in the preceding Methods section and used to transfect normal, human cultured keratinocytes. The cultured keratinocytes transfected with 1α-OHase gene were incubated with varying concentrations of 25(OH)D₃. As a negative control, cultured keratinocytes that had not been transfected were exposed to the same concentrations of 25(OH)D under identical conditions at the same time. If the cells transfected with the 1α-OHase gene increased the 1α-OHase activity in the cells, the cells would be more efficient in converting 25(OH)D to 1,25(OH)₂D which, in turn, would result in higher concentrations within the cell, and therefore, have a more dramatic effect on decreasing the cells' proliferative activity. As can be seen in FIG. 6, the keratinocytes transfected with the 1α-OHase gene had a more marked decrease in proliferative activity to the same concentration of 25(OH)D compared to the non-transfected control keratinocytes. This occurred at concentrations from 10⁻⁹ to 10⁻⁷M. There was a significant (p<0.01) 53.7% reduction in the proliferative activity of the cells transfected with the 1α-OHase gene and incubated with 25(OH)D at 10⁻⁷ M compared to untransfected keratinocytes.

It should be understood that the examples and embodiments described herein are for illustrative purposes only and that various modifications or changes in light thereof will be suggested to persons skilled in the art and are to be included within the spirit and purview of this application and the scope of the appended claims. All publications, patents and patent applications cited herein are incorporated by reference herein in the entirety.

1. A method for inhibiting tumor cells, while reducing the risk of UV radiation exposure or vitamin D toxicity, said method comprising the step of administering to a patient a composition comprising an effective amount of a metabolic precursor of 1,25-dihydroxyvitamin D to increase levels of 1,25-dihydroxyvitamin D in said tumor cells in a target organ wherein the tumor cells have a hydroxylase enzyme for synthesizing 1,25-dihydroxyvitamin D from said metabolic precursor.

2. The method of claim 1 wherein said metabolic precursor is 25-hydroxyvitamin D.

3. The method of claim 1 wherein said hydroxylase enzyme is 25-hydroxyvitamin D-1α-hydroxylase.

4. The method of claim 1 wherein said tumor cells are selected from the group consisting of prostate cancer cells, breast cancer cells, skin cancer cells, pancreatic cancer cells, colon cancer cells, lung cancer cells, leukemia cells, and lymphoma cells.

5. The method of claim 1 wherein said tumor cells are prostatic cancer cells.

6. The method of claim 1 wherein the effective amount of said metabolic precursor is an amount which results in intra-target organ cell levels of said metabolic precursor between about 25 and about 250 nmol/L.

7. The method of claim 1 wherein said metabolic precursor is administered as a composition comprising said metabolic precursor, or a salt, isomer, or derivative thereof, and a pharmaceutically acceptable carrier.

8. A method for inhibiting cancer cells, while reducing the risk of UV radiation exposure or vitamin D toxicity, said method comprising the step of administering to a patient a composition comprising an effective amount of a metabolic precursor of 1,25-dihydroxyvitamin D to increase levels of 1,25-dihydroxyvitamin D in cancer cells in a target organ wherein the target organ cancer cells have a hydroxylase enzyme for synthesizing 1,25-dihydroxyvitamin D from said metabolic precursor.

9. The method of claim 8 wherein said metabolic precursor is 25-hydroxyvitamin D.

10. The method of claim 8 wherein said hydroxylase enzyme is 25-hydroxyvitamin D-1α-hydroxylase.

11. The method of claim 8 wherein said cancer cells are selected from the group consisting of prostate cancer cells, breast cancer cells, skin cancer cells, colon cancer cells, pancreatic cancer cells, lung cancer cells, leukemia cells, and lymphoma cells.

12. The method of claim 8 wherein said tumor cells are prostatic cancer cells.

13. The method of claim 8 wherein the effective amount of said metabolic precursor is an amount which results in intra-target organ cell levels of said metabolic precursor between about 25 and about 250 nmol/L.

14. The method of claim 8 wherein said metabolic precursor is administered as a composition comprising said precursor or a salt, isomer, or derivative thereof, and a pharmaceutically acceptable carrier.

15. A method for treating benign prostatic hyperplasia in an animal, while reducing the risk of UV radiation exposure or vitamin D toxicity, said method comprising the step of administering to the animal a composition comprising an effective amount of a metabolic precursor of 1,25-dihydroxyvitamin D to increase levels of 1,25-dihydroxyvitamin D in prostatic cells having a hydroxylase enzyme for synthesizing 1,25-dihydroxyvitamin D from said metabolic precursor.

16. The method of claim 15 wherein said metabolic precursor is 25-hydroxyvitamin D.

17. The method of claim 15 wherein said hydroxylase enzyme is 25-hydroxyvitamin D-1α-hydroxylase.

18. The method of claim 15 wherein the effective amount of said metabolic precursor is an amount which results in levels of said metabolic precursor in prostate cells of between about 25 and about 250 nmol/L.

19. The method of claim 15 wherein said metabolic precursor is administered as a composition comprising said precursor or a salt, isomer, or derivative thereof and a pharmaceutically acceptable carrier.
